(19) United States
(12) Patent Application Publication
(10) Pub. No.: US 2003/0232101 A1
(43) Pub. Date: Dec. 18, 2003

(54) Topical formulations of resorcinols and cannibinoids and methods of use

(75) Inventor: Craig R. Travis, South Miami, FL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Immugen Pharmaceuticals, Inc., South Miami, FL

(21) Appl. No.: 10/391,845

(22) Filed: Mar. 18, 2003

Related U.S. Application Data
(60) Provisional application No. 60/365,329, filed on Mar. 18, 2002.
(60) Provisional application No. 60/365,700, filed on Mar. 19, 2002.

(51) Int. Cl. A61K 31/353; A61K 35/78

(52) U.S. Cl. 424/776; 514/455; 514/454

(57) ABSTRACT

In one aspect, the invention provides a method for preventing the transmission of HIV from one individual to another. In accordance with the method, a pharmacologically-acceptable composition including at least one resorcinol derivative compound and/or cannabinoid (e.g., cannabinol derivatives, Δ8-THC derivatives, cannabichromene derivatives, cannabidiol derivatives, cannabigerol derivatives) (including combinations thereof) is administered topically to a first individual harboring HIV, or to a second individual at risk of infection with HIV, proximate in time with contact between the first individual and the second individual. The invention also provides topical formulations of at least one resorcinol and/or cannabinoid and water insoluble polymers as hydrogels.

### TECHNICAL FIELD OF THE INVENTION

The present invention pertains to the prevention of HIV infection by means of interpersonal transmission.

### BACKGROUND OF THE INVENTION

Sexual transmission is the leading modality in the spread of HIV disease globally with women representing the greatest number of individuals contracting the virus through an infected male partner. It is now accepted that sexually transmitted diseases (STDs) put people at greater risk of becoming infected with HIV with women suffering the greatest consequence of unprotected sex with highly promiscuous partners. Clearly there is a need for a topical agent which a woman can use prior to a sexual encounter which will provide her with protection against becoming infected with HIV. Ideally such an agent should be able to have effect within a short period of application without disturbing the normally protective vaginal flora and without any particular offensive characteristics such as odor, irritation or alteration of the mucosal barrier making it more susceptible to micro abrasions. Such an agent should ideally be long lasting and not washed away by vaginal douches and can be reapplied subsequently without disturbing the sexual encounter whether risk is involved or not. Interpersonal transmission such as maternal to fetus during childbirth and breastfeeding is another event which can be prevented by pretreating the expectant mother prior to delivery and swabbing an infants mouth (mucosal surface) prior to breast feeding.

The retroviral Human Immunodeficiency Viruses 1 and 2 (HIV) are the most common causative agents of AIDS. In order to cause infection, HIV needs to gain access to the host's immune system. Except for blood transfusions and needle exposure with infected blood, the most common mode of transmission is sexual either through the vaginal epithelium in a heterosexual encounter or anal rectal in a homosexual one. The mucosal surface in the vagina presents a significant barrier to HIV transmission. Although the vaginal epithelium may be effective it is not an absolute barrier to HIV-1 infection since the virus may have the ability to sequester itself in the epithelial cells for presentation to the intraepithelial and lamina propria immune cells. Studies have shown that the virus first appears in the layer immediately below the lamina propria, which provides the fluid and moisture to the epithelium. Although vaginal epithelium is different from the epithelium of the rectum, the endocervical epithelium is similar to the columnar epithelium of the rectum and various factors promoting the earliest events take place there. Here, through direct contact with these surfaces either by penetration of the abraded mucosa or by invasion of the surface in association with an infectious co-factor, the virus not only gains entry into the epithelium but also encounters the first of highly specialized antigen presenting cells (APC) known as dendritic cells (DC). The first APCs to become exposed to the virus are the Langerhans cells which interdigitate among the epithelial cells. Langerhans cells are immature dendritic cells lacking the costimulatory molecules CD80 and CD86 which become expressed later as they migrate to the lymph nodes and mature. However, they do possess functioning CCR5 receptors and are productive of M-trophic HIV virus which may augment plasma viremia and spread the virus to T cells. Additionally, Langerhans cells express CD24 or HSA which is a molecule involved in T cell proliferation.

Although the genital tract can be infected with either SI/X4 or NSI/R5 variants, the macrophage-tropic NSI predominates. Interestingly enough CXCR4 is expressed in the dendritic cells to much greater degree than CCR5 suggesting that mucosal T cells and macrophages are more likely to become infected with R-5 trophic virus. It has also been shown recently that the full range of mucosal mononuclear cells (MMC) can be infected by the R-5 (M-tropic) HIV-1 strain following sexual exposure. These include T lymphocytes which express CCR5 to a greater extent than found in the blood compartment yet also maintain a high level of expression of CXCR4. Macrophages expressing CCR5 are also present. However, genital T cells expressing both CXCR4 and CCR5 are susceptible to both R-5 and X-4 strains of the virus. The observation of clusters of T cells with CD14+ macrophages suggests that macrophages could be acting as long term reservoirs and their role in antigen processing and presentation could shape the repertoire of the immune response after infection. Another study confirmed that subendothelial macrophages are the prime target cells for HIV-1 infection in genital tract mucosal tissue in organ culture and that there was no evidence of infection within the cervical epithelium. Once infected by the M-trophic HIV virus these professional antigen presenters form heterlogous syncitia or bond with CD4+ T-lymphocytes in the internal iliac lymph nodes drained by the afferent lymphatics of the anal/genital mucosal sites.

Following binding, the virus fuses with the cell membrane and is internalized. Within the cell, it produces a reverse transcriptase which transcribes its genomic RNA to DNA. The reverse HIV transcript is then integrated into the cellular DNA where it exists for the life of the cell as a “provirus.” The provirus can remain latent for an indefinite period of time, or it can be activated to transcribe mRNA and genomic RNA, leading to protein synthesis, assembly, new virion formation, budding of virus from the cell surface, and cell death.

The use of a barrier device such as a condom can prevent STDs but requires cooperation of the male partner and its lack of acceptance is without doubt its failing. Moreover, it has been demonstrated that detergent spermicides, such as nonoxynol-9 (N-9) actually increase the risk of HIV transmission. As such there is a need for an agent(s) which are not associated with irritation or ulceration of either the cervicovaginal or penile epithelium that will serve as a topical microbicide if the problem of the transmission of STDs is to be limited.

### BRIEF SUMMARY OF THE INVENTION

In one aspect, the invention provides a method for preventing the transmission of HIV from one individual to another. In accordance with the method, a pharmacologically-acceptable composition including at least one resorcinol derivative compound and/or cannabinoid (e.g., cannabinol derivatives, Δ8-THC derivatives, cannabichromene derivatives, cannabidiol derivatives, cannabigerol derivatives) (including combinations thereof) is administered topically to a first individual harboring HIV, or to a second individual at risk of infection with HIV, proximate in time with contact between the first individual and the second individual. The invention also provides topical formulations of at least one resorcinol and/or cannabinoid and water insoluble polymers as hydrogels. These and other advantages of the present invention, as well as additional inventive features, will be apparent from the accompanying Figures and the following detailed description.

### DESCRIPTION OF THE FIGURES

FIG. 1 graphically compares the IC₅₀ values measured in peripheral blood mononuclear cells using time of addition assays.

FIG. 2 graphically presents the level of reverse transcription observed using time-of-addition studies using AZT.

FIG. 3 graphically presents the relative IC₅₀ measured in peripheral blood mononuclear cells using time of addition assays during the interval from virus entry to completion of reverse transcription.

FIG. 4 graphically presents the maximal suppression of HIV replication measured in peripheral blood mononuclear cells using time of addition assays during the interval from virus entry to completion of reverse transcription.

### DETAILED DESCRIPTION OF THE INVENTION

At least one compound for use in the invention can be a resorcinol derivative (e.g., a 5-alkyl or 3-alkyl or -acyl resorcinol). In a preferred embodiment, at least one compound within the pharmacologically-acceptable composition can be a 5-alkyl-resorcinol derivative. Such compounds are advantageous for use in the inventive method as they generally exhibit low cytoxicity. The manufacture and formulation of compounds suitable for use in the inventive method and compositions is known in the art (see, e.g., U.S. Pat. Nos. 5,859,067, 6,274,635 and published international patent application WO 00/56303, which are incorporated herein by reference). Particularly preferred alkyl-resorcinol derivatives have the following formula:

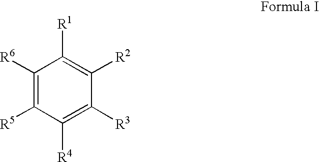

wherein,

R¹, R³, R⁵, and R⁶ can optionally be —COR¹, —COR³, —COR⁵, and/or —COR⁶, respectively, and preferably R³ is —COR³, and wherein R can otherwise be as follows:

R¹ is:

a) H,

b) a C₁₋₄alkyl group or ester thereof,

c) COOH,

d) OH,

e) a O-C₁₋₅ alkyl (preferably OCH₃) or alkanoyl, optionally substituted by mono- or di- methylamino or ethylamino groups,

f) a O—CO—C₃₋₁₀ alkyl group containing a carboxyl or amino group,

g)

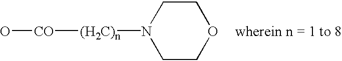

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen (e.g., fluorine, bromine, iodine, astatine);

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl (e.g., -pentyl, -hexyl, -heptyl, -octyl, or -nonyl), alkenyl, alkynyl, group, or mixture thereof, optionally substituted with at least one halogen (e.g., halogen terminal group or even dihalogen),

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄alkyl, CONH₂, CONHC₁₋₄alkyl, or CON(C₁₋₄alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁴ is:

a) H or halogen (preferably bromine)

b) OH, or

c) C₁₋₆ alkoxyl or carboxyl;

R⁵ is

a) H,

b) a C₁₋₄ alkyl group,

c) COOH,

d) OH, or OCH₃,

e) a O—C₁₋₅ alkyl (ether) or alkanoyl, optionally substituted with at least one mono- or di- methylamino or ethylamino group, or

f) a lactone; and

R⁶ is:

a) H or OH;

b) C₁₋₄ alkyl (preferably ethyl), alkenyl, alkynyl, group, or mixture thereof,

c) O—C₁₋₄ alkyl, alkenyl, alkynyl, group, or mixture thereof, or

d) a pryenyl, gerenyl, or farnesyl group, optionally substituted at any position with one or more halogens,

e) (W)ₘ—Y-(Z)ₙ, wherein

W is a C₅₋₁₂ alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄alkyl, CONH₂, CONHC₁₋₄alkyl, or CON(C₁₋₄alkyl)₂, wherein each C₁₋₄ alkyl on the amide nitrogen can be the same or different, and wherein

m and n are the same or different, and each is either 0 or 1,

f) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄alkyl)₂, wherein each C₁₋₄ alkyl on the amide nitrogen can be the same or different,

g) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

h) CH(CH₃)CO₂H, CH₂COOH, or —OCOCH₃.

Compounds according to Formula I preferably include a lactone, H, OH or OCH₃, —CH(CH₃)CO₂H, or —OCOCH₃ as R¹ substituents. Preferred substituents at R² are hydrogen, halogen (most preferably fluorine) hydroxyl, COOH, or methoxyl groups. Preferred substituents at R⁴ include H or a halogen (most preferably bromine). Preferred substituents at R⁵ include a lactone, H, OH, and OCH₃. Preferred substituents at R include H, OH, ethyl, CH(CH₃)CO₂H, CH₂COOH, and —OCOCH₃. Where compounds of formula I are included, preferably R⁶ is methyl or ethyl. A more preferred compound according to Formula I has hydroxyl substituents at R¹, R⁵, and a methyl substituent at R⁶; even more preferably, the compound has a third hydroxyl substituent at R². Preferred substituents at R³ are discussed elsewhere herein; however, the invention provides compounds according to Formula I, wherein

R³ is:

a) (W)ₘ—Y-(Z)ₙ, wherein

W is a C₅₋₁₂ alkyl, alkenyl, alkynyl (e.g., 2′-ynyl, 3′-ynyl or 4′-ynyl), group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl (e.g., 2′-ynyl, 3′-ynyl or 4′-ynyl), group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different,

wherein at least one of W and Z includes a branched chain and wherein m and n are the same or different, and each is either 0 or 1,

b) a terminally-branched (e.g., terminal dimethyl) C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a terminally-branched C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different.

Particularly preferred R³ substituents include C₅-C₁₂ alkynes, and particularly preferred groups also include di- or tri-methyl terminal groups. A most preferred substituent at R³ is a dimethylheptyl, particularly 1′S, 2′SR, and also preferably with terminal halogen (or dihalogen) substituents, and another preferred substituent is 5,5-diimethyl hex(1-ene)(3-yne)yl (e.g., compound Ii). Many such compounds exhibit antineoplastic activity and can be employed as such, as described herein. While any such compounds can be included within the composition in accordance with the inventive method, some preferred compounds are as follows:

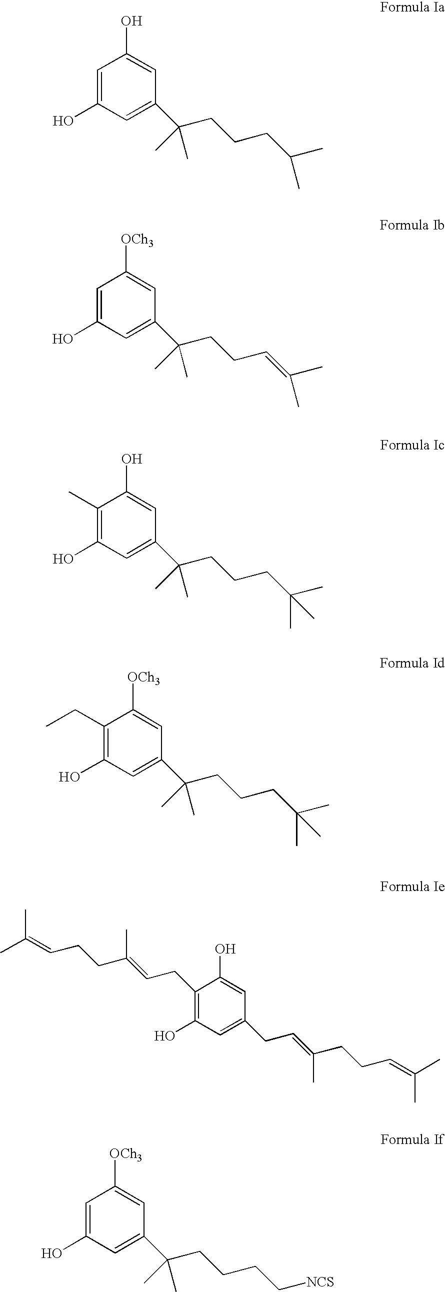

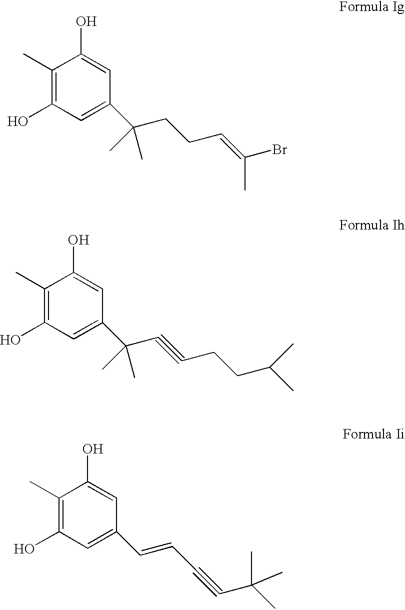

As mentioned, compounds according to Formula I can have gerenyl substituents at R⁶. In this regard, the compound for use in the context of the present invention composition can be cannabigerol or a derivative thereof having the following formula:

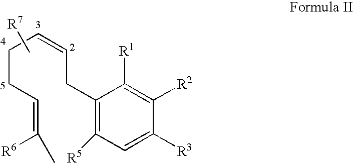

wherein:

R¹ is:

a) H,

b) a C₁₋₄ alkyl group or ester thereof,

c) COOH,

d) OH,

e) a O—C₁₋₅ alkyl (preferably OCH₃) or alkanoyl, optionally substituted by mono- or di- methylamino or ethylamino groups,

f) a O—CO—C₃₋₁₀ alkyl group containing a carboxyl or amino group,

g)

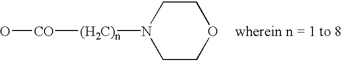

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁵ is

a) H,

b) a C₁₋₄ alkyl group,

c) COOH,

d) OH, or OCH₃,

e) a O—C₁₋₅ alkyl (ether) or alkanoyl, optionally substituted with at least one mono- or di- methylamino or ethylamino group, or

f) a lactone; and

R⁶ is:

a) hydrogen,

b) C₁₋₆ alkoxy , C₁₋₆ alkylthio, C₁₋₆ alkyl (preferably ethyl), or C₁₋₆ haloalkyl,

c) CN,

d) CO₂H,

e) CO₂-C₁₋₄ alkyl,

f) C(Y)(Z)-OH,

g) C(Y)(Z)-O—C₁₋₄ alkyl, or

h) C₁₋₆ alkyl-CO₂—Y,

wherein Y and Z are each independently H or C₁₋₆ alkyl,

R⁷ is:

a) hydroxy (preferably β-hydroxy) or lactone,

b) halo,

c) C₁₋₆ alkoxy, C₁₋₆ alkylthio, C₁₋₆ alkyl, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k) ═O or ═S,

wherein Y and Z are each independently H or C₁₋₆ alkyl, and wherein R⁷ can be at any of positions 2-5.

Compounds according to Formulas I and II can be synthesized using known procedures from commercially available starting materials (see, e.g., Dominianni et al., J. Org Chem., 42, 344-46 (1977); Baek et al., Arch. Pharm. Res., 19, 228-30 (1996); Guthrie et al., J. Org. Chem. 47, 2369-76 (1982)). For example, acid catalyzed condensation of 2,6-dimethoxyphenol with OH—R³ can produce a 4-alkylphenol intermediate. Conversion of the phenolic group to the diethylphosphate ester followed by reduction with lithium metal in liquid ammonia can then produce a dimethoxybenzene derivative. Mono- or didemethylation of this compound (e.g., with boron tribromide) can then yield the desired methoxyphenol and/or resorcinol (Formula I), respectively. Compounds of Formula I having alkyl substituents at R⁶ can be prepared, for example, first by lithiation of the dimethoxybenzene derivative at R⁶ (e.g., in the presence of Bu/THF) and subsequent exposure to an alkylating agent (e.g., methyl or ethyl iodide or sulfate). Mono- or didemethylation of this compound (e.g., with boron tribromide) can then yield the desired methoxyphenol and/or resorcinol (Formula I), respectively, having the alkyl substituents at R⁶. Compounds of Formula II can be prepared, for example, by acid catalyzed condensation of a methoxyphenol and/or resorcinol (Formula I) having a desired substituents at R³ with geraniol (e.g., in the presence of BF₃, Et₂O, silica, and CH₂Cl₂). Of course, these compounds can be synthesized by other appropriate methods, many of which are known in the art.

In another embodiment, at least one compound for use in the context of the present invention can be cannabinol or a derivative thereof (e.g., Δ8-tetrahydrocannabinol, Δ9-tetrahydroxcannabinol, or derivatives thereof. Other preferred cannabinol derivatives can have the following formula:

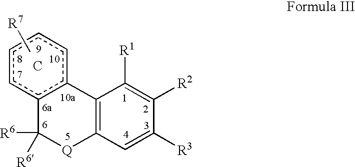

wherein,

R¹ is:

a) H,

b) a C₁₋₄ alkyl group or ester thereof,

c) COOH,

d) OH,

e) a O—C₁₋₅ alkyl (preferably OCH₃) or alkanoyl, optionally substituted by mono- or di- methylamino or ethylamino groups,

f) a O—CO—C₃₋₁₀ alkyl group containing a carboxyl or amino group,

g)

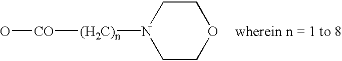

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁶ and R^{6′} together form ═O or ═S, or each is independently selected from the group consisting of:

a) hydrogen,

b) C₁₋₆ alkoxy, C₁₋₆ alkylthio, C₁₋₆ alkyl, or C₁₋₆ haloalkyl,

c) CN,

d) CO₂H,

e) CO₂—C₁₋₄ alkyl,

f) C(Y)(Z)-OH,

g) C(Y)(Z)-O—C₁₋₄ alkyl, and

h) C₁₋₆ alkyl-CO₂—Y,

wherein Y and Z are each independently H or C₁₋₆ alkyl,

R⁷ is:

a) hydroxy or lactone,

b) halo,

c) C₁₋₆ alkoxy, C₁₋₆ alkylthio, C₁₋₆ alkyl, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k) ═O or ═S,

wherein Y and Z are each independently H or C₁₋₆ alkyl;

Q is:

a) O or S, or

b) N—W, wherein

W is:

i) hydrogen,

ii) C₁₋₆ alkoxyalkyl, C₁₋₆ alkyl, or C₁₋₆ haloalkyl

iii) OC₁₋₆ alkyl, or OC₁₋₆ haloalkyl,

iv) CN,

v) C₁₋₆ alkyl,

vi) C(Y)(Z)C₁₋₄ alkyl, or

vii) C₁₋₆ alkyl-CO₂-Z,

wherein Y and Z are each independently H or C₁₋₆ alkyl.

Preferably R¹ in Formula III is H, O—C₁₋₄ alkyl (more preferably methoxy) or a hemi ester of succinic acid, malonic acid or the alaninate ester of alanine and salts thereof. In another preferred embodiment, R¹ and R² together comprise a substituent of the formula —O(CH₂)₃₋₅—, wherein R¹ and R², together with the carbon atoms to which they are bonded, comprise a ring where at least one hydrogen atom thereof is optionally substituted with a halogen (e.g., an O, 2 propano ring). Furthermore, where R² Formula III is a halogen, preferably it is iodo. Preferably, R⁶ and R^{6′} together form ═O or each are methyl, ethyl, or methoxy.

While R⁷ can be at any of positions 7-10 of ring C, preferably it is at position 9 of the ring. Also, in some embodiments, R⁷ preferably is electronegative (e.g., COOH, halogen, β-hydroxy, or lactone.), while in others, it can be substituted with either a lactone or a □-hydroxy group.

Ring C in Formula III can be any of the following (the dashed lines representing a double bond at either the Δ6a-10a, Δ8-9, or Δ9-10 position):

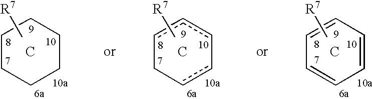

However, preferably the ring is aromatic. In such compounds, R⁷ preferably is electronegative and more preferably is on C9. Furthermore, for such embodiments, R¹ preferably is other than OH and preferably is deoxy, an ester, or an ether. Exemplary cannabinol derivative compounds include:

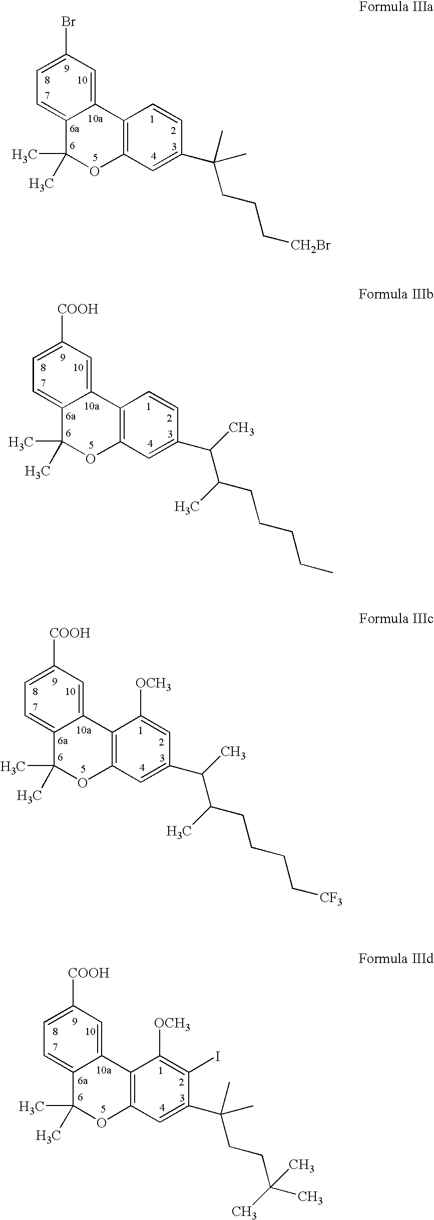

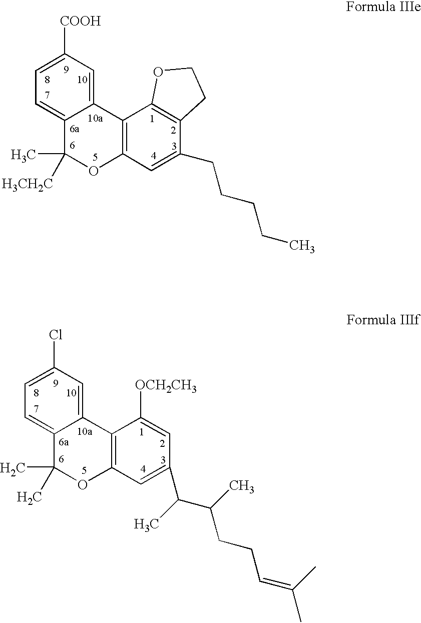

Another preferred compound according to Formula III is a derivative of delta-8 tetrahydrocannabinol, in which R¹ is an acetate, R⁶ is a lactone, and R⁷is COOH (exemplary species of which are described, for example, in Rhee et al. J. Med. Chem., 40, 3228-33 (1997)).

Many compounds according to Formula III are well known, and others can be manufactured in accordance with published methods (see, for example, International Patent Application WO99/20268 (Burstein), and U.S. Pat. Nos. 2,509,386 (Adams), 3,799,946 (Loev), 3,856,821 (Loev), 3,897,306 (Vidic et al.), 4,064,009 (Fukada et al.), 4,087,545 (Archer et al.), 4,142,139 (Bindra), 4,309,545 (Johnson), 4,599,327 (Nógrádi et al.), 4,833,073 (McNally et al.), 4,876,276 (Mechoulan et al.), 4,973,603 (Burstein), 5,338,753 (Burstein et al.), 5,389,375 (ElSohly), 5,440,052 (Makriyannis et al.), 5,605,906 (Lau), and 5,635,530 (Mechoulam et al.); and Charalambous et al., Pharm. Biochem. Behav., 40, 509-12 (191), Gareau et al., Bioorg. Med. Chem. Lett., 6(2), 189-94 (1996), Griffin et al., Br. J. Pharmacol., 126, 1575-84 (1999), Huffman et al., Bioorg. Med. Chem. Lett., 6, 2281-88 (1998), Lemberger et al., Clin. Pharmacol. Ther., 18(6), 720-26 (1975), Loev et al., J. Med. Chem., 16(11), 1200-06 9 (1973), Loev et al., J. Med. Chem., 17(11), 1234-35 (1974), Martin et al., Pharm. Biochem. Behav., 46, 295-301 (1993), Papahatjis et al., J. Med. Chem., 41(7), 1195-1200 (1998), Pars et al., J. Med Chem., 19(4), 445-53 (1976), Pertwee et al., Pharmacol. Ther., 74(2), 129-80 (1997), Razdan et al., J. Med. Chem., 19(4), 454-60 (1976), Razdan, Pharmacol. Reviews, 38(2) 75-149 (1980), Reggio et al., J. Med. Chem., 40(20), 3312-18 (1997), Reggio et al., Life Sci., 56(23/24), 2025-32 (1995), (Ross et al., Br. J. Pharmacol., 126, 665-72 (1999), Thomas et al., J. Pharm. Exp. Ther., 285(1), 285-92 (1998), Wiley et al., J. Pharm. Exp. Ther., 285(1), 995-1004 (1998), Winn et al., J. Med. Chem., 19(4), 461-71 (1976), and Xie et al., J. Med Chem., 41, 1-67-74 (1998)).

In the preferred embodiment wherein ring C of Formula III is aromatic, such compounds additionally can be manufactured by aromatizing an appropriate tetrahydrocannabinol (THC) derivative molecule by known methods (see, e.g., Adams et al., J. Am. Chem. Soc., 62, 23401 (1940); Ghosh et al., J. Chem. Soc., 1393 (1940); and Adams et al., J. Am. Chem. Soc., 70, 664 (1948)). For example, aromatization of such compounds can occur by heating the compound with sulfur at about 238-240° C., under a nitrogen atmosphere, for about 4 hours (Rhee et al., J. Med Chem., 40(20), 3228-33 (1997)). Other suitable methods include aromatization using a catalyst (e.g., palladium on carbon) or a chemical dehydrogenating agent (e.g., 2,3-dichloro-5,6-dicyanoquinone) (see, for example, U.S. Pat. No. 3,799,946 (Loev)).

As mentioned, in some applications, particularly where at least one of the compounds within the composition is a cannabinol derivative, it is desirable to mitigate potentially deleterious psychoactivity attributed to some such compounds. As an alternative to employing non-psychoactive cannabinol derivatives (e.g., selective CB2 agonists) within the composition, other pharmacologically-active agents can be employed in addition to mitigate psychoactive effects. For example, as some of the aforementioned compounds might exert some activity on CB1 receptors, it is often desirable to adjunctively administer a selective CB1 antagonist to the patient. Many suitable selective CB1 antagonists are known in the art (Rinaldi-Carmona et al., FEBS Lett., 350, 240-44 (1994), see also U.S. Pat. Nos. 5,624,941 (Barth et al.), 5,747,524 (Cullinan et al.), 5,925,768 (Barth et al.)). SR-124171-6A is a particularly potent, and theretofore preferred, selective CB1 antagonist for use in the inventive method. Other preferred selective CB1 antagonists are cannabidiol and its derivatives (see, e.g., U.S. Pat. No. 2,304,669 (Adams); Razdan et al., Pharmacol. Reviews, 38(2), 75-149 (1986); Reggio et al., Life Sci., 56(23-24), 2025-32 (1995)), as these potently antagonize the CB1 receptor. In addition to antagonizing CB1, cannabidiol and many of its derivatives also advantageously attenuate the cytochrome P₄₅₀ system in the liver, leading to enhanced bioavailability of other compounds within the composition (e.g., Bornheim et al., Chem. Res. Toxicol., 11, 1209-16 (1998)). In this regard, in some embodiments of the inventive method, at least one compound within the pharmacologically-acceptable composition is cannabidiol or a derivative thereof. Preferred cannabidiol derivatives can, for example, have the following formula:

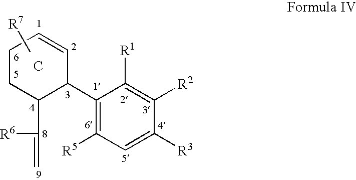

wherein:

R¹ is:

a) H,

b) a C₁₋₄ alkyl group or ester thereof,

c) COOH,

d) OH,

e) a O—C₁₋₅alkyl (preferably OCH₃) or alkanoyl, optionally substituted by mono- or di- methylamino or ethylamino groups,

f) a O—CO—C₃₋₁₀ alkyl group containing a carboxyl or amino group,

g)

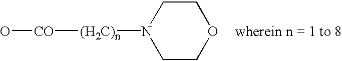

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) RX and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R² together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄alkyl, CONH₂, CONHC₁₋₄alkyl, or CON(C₁₋₄alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁵ is

a) H

b) a C₁₋₄ alkyl group

c) COOH

d) OH, or

e) a O—C₁₋₅ alkyl (ether) or alkanoyl, optionally substituted with at least one mono- or di- methylamino or ethylamino group;

R⁶ is:

a) hydrogen,

b) C₁₋₆ alkoxy, C₁₋₆ alkylthio, C₁₋₆ alkyl, or C₁₋₆ haloalkyl,

c) CN,

d) CO₂H,

e) CO₂—C₁₋₄ alkyl,

f) C(Y)(Z)-OH,

g) C(Y)(Z)-O—C₁₋₄ alkyl, or

h) C₁₋₆ alkyl-CO₂—Y,

wherein Y and Z are each independently H or C₁₋₆ alkyl,

R⁷ is:

a) hydroxy or lactone,

b) halo,

c) C₁₋₆ alkoxy, C₁₋₆ alkylthio, C₁₋₆ alkyl, C₁₋₆ carboxy, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k) ═O or ═S,

wherein Y and Z are each independently H or C₁₋₆ alkyl, and wherein R⁷ can be at any of positions 1, 2, 5, or 6 of ring C.

Another preferred compound for use in the context of the present invention is cannabichromene or a derivative thereof. Preferably cannabichromene derivatives can have, for example, the following formula:

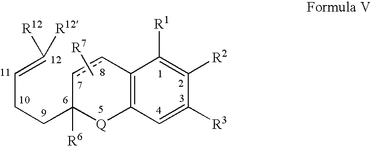

wherein,

R¹ is:

a) H,

b) a C₁₋₄ alkyl group or ester thereof,

c) COOH,

d) OH,

e) a O—C₁₋₅alkyl (preferably OCH₃) or alkanoyl, optionally substituted by mono- or di- methylamino or ethylamino groups,

f) a O—CO—C₃₋₁₀ alkyl group containing a carboxyl or amino group,

g)

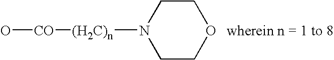

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆alkyl, C₁₋₆alkoxy, C₁₋₆alkylthio, CN, CF₃, CO₂H, or CO₂C₁₋₄alkyl, CONH₂, CONHC₁₋₄alkyl, or CON(C₁₋₄alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄alkyl, CONH₂, CONHC₁₋₄alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁶ is selected from the group consisting of:

a) hydrogen,

b) hydroxy or lactone,

c) halo,

d) C₁₋₆ alkoxy, C₁₋₆ alkylthio, C₁₋₆ alkyl, or C₁₋₆ haloalkyl,

e) CN,

f) N₃,

g) CO₂H,

h) CO₂-C₁₋₄ alkyl,

i) C(Y)(Z)-OH,

j) C(Y)(Z)-O—C₁₋₄ alkyl, and

k) C₁₋₆ alkyl-CO₂—Y,

wherein Y and Z are each independently H or C₁₋₆ alkyl,

R⁷ is selected from the group consisting of:

a) hydrogen,

b) hydroxy or lactone,

c) halo,

d) C₁₋₆ alkoxy, C₁₋₆ alkylthio, C₁₋₆ alkyl, or C₁₋₆ haloalkyl,

e) CN,

f) N₃,

g) CO₂H,

h) CO₂-C₁₋₄ alkyl,

i) C(Y)(Z)-OH,

j) C(Y)(Z)-O—C₁₋₄ alkyl,

k) C₁₋₆ alkyl-CO₂—Y, and

l) ═O or ═S;

wherein Y and Z are each independently H or C₁₋₆ alkyl,

R¹² and R^{12′} together form ═O or ═S, or each is independently selected from the group consisting of:

a) hydrogen,

b) hydroxy or lactone,

c) halo,

b) C₁₋₆ alkoxy, C₁₋₆ alkylthio, C₁₋₆ alkyl, or C₁₋₆ haloalkyl,

c) CN,

d) N₃,

d) CP₂H,

e) CO₂—C₁₋₄ alkyl,

f) C(Y)(Z)-OH,

g) C(Y)(Z)-O—C₁₋₄ alkyl, and

h) C₁₋₆ alkyl-CO₂—Y,

wherein Y and Z are each independently H or C₁₋₆ alkyl,

Q is:

a) O or S, or

b) N—W, wherein

W is:

i) hydrogen,

ii) C₁₋₆ alkoxyalkyl, C₁₋₆ alkyl, or C₁₋₆ haloalkyl

iii) OC₁₋₆ alkyl, or OC₁₋₆ haloalkyl,

iv) CN,

v) C₁₋₆ alkyl,

vi) C(Y)(Z)C₁₋₄ alkyl, or

vii) C₁₋₆ alkyl-CO₂-Z,

wherein Y and Z are each independently H or C₁₋₆ alkyl.

Many cannabichromene derivatives are known, and others can be synthesized using methods that are known in the art (see, e.g., U.S. Pat. No. 4,315,862).

In addition to having the indicated substituents, R³ in any of formulas I-V preferably is:

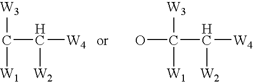

wherein W₁ is H, methyl, or ethyl, wherein W₂ and W₃ are each independently H or methyl, wherein at least one of W₁, W₂, and W₃ is other than H and/or halogenated, and wherein W₄ is a C₁₋₄ alkyl or haloalkyl, optionally substituted with an aromatic ring. Preferably, R³ is a branched C₆₋₁₂ alkyl group containing at least one double bond (more preferably at position C₄-C₁₀), and preferably the chain has an odd number of carbon atoms. More preferably, R³ is terminally branched or contains a terminal double bond, and the invention provides compounds according to Formulas I-V having such substituents. More preferably, R³ preferably is dimethylheptyl (DMH) (e.g., 1′,1′ DMH or 1′R, 2′S DMH), dimethylhexyl, or dimethylpentyl. For example, R³ can be a di- tri- or tetramethylpentyl, -hexyl, or -heptyl, etc., chain (e.g., 1,1,5-trimethylhexyl, 1,1,5,5-tetramethylhexyl, or 1,1,5-trimethyl-hept-4-enyl). In some instances, the R³ substituent can have bulky terminal moieties, for example, methyl, dimethyl, (CH₂)₁₋₆—CON(CH3)2, or C6-12 haloalkyl with halogenated terminal carbon atoms (preferably bromine, fluorine and iodine).

In the context of this invention, halogenated alkanes, alkenes, and alkynes can have any number of halogen substitutions. In a preferred embodiment, the halogenated alkane, alkene, or alkyne has at least one halogen on a terminal carbon atom (e.g., CX₁₋₃, wherein X is halogen). Alkyl groups (as well as alkenes and alkynes) can be straight chain or branched. Moreover, the compounds can exist as a single stereoisomer or a mixture of stereoisomers (e.g., a racemic mixture), or a single geometric isomer (e.g., E, Z, cis or trans) or a mixture of geometric isomers, all of which are within the scope of the invention.

In one embodiment, the invention provides a method for preventing the transmission of HIV from one individual (e.g., a first HIV-infected individual) to another (e.g., a second individual at risk of HIV infection). In accordance with the method, a pharmacologically-acceptable composition including at least one resorcinol derivative compound and/or cannabinoid (e.g., cannabinol derivatives, Δ⁸-THC derivatives, cannabichromene derivatives, cannabidiol derivatives, cannabigerol derivatives) is administered topically. The resorcinol derivative compound and/or cannabinoid can be one or a combination of such compounds, such as descried above.

In conjunction with the inventive method, the resorcinol derivative compound(s) and/or cannabinoid(s) can be applied topically to the surface of the first or the second individual, or even to both individuals. In this regard, the resorcinol derivative compound(s) and/or cannabinoid(s) can be applied to the skin, mucous tissue, epithelia lining oral cavities or other cavities, or any other suitable portion of the surface of one or both of the individuals. The compound acts to prevent HIV infection of the second individual at risk of HIV infection by virtue of contact with the first individual.

The method also can be employed to protect individual at risk of HIV infection from HIV infection from an item contaminated with HIV. In this context, the item can be any item that can be contaminated with HIV, such as a needle, blood or blood product, a barrier contraceptive, or other device. To assist in protecting the individual at risk of HIV infection from HIV upon contact with such a device, the resorcinol derivative and/or cannabinoid compound(s) is applied to the individual and/or to the item, proximate in time to contact between the individual and the item.

The inventive method is particularly effective in combating HIV disease in humans, which is transmitted primarily by sexual intercourse. Indeed, applications of the inventive method in which the composition is administered to mucosal tissue (e.g., vaginal or rectal tissue), can retard the uptake of the virus through such tissues, thus reducing the incidence of primary infection. Thus, the invention provides a method of preventing the transmission of HIV. Furthermore, the highly lipophilic nature of the drugs ensures its non-specific binding to vaginal, cervical and colonic epithelium providing a barrier against further transmission through the interdigitating Langerhans cells or dendritic cells which would also be exposed to the drug.

In the practice of the inventive method, typically, the alkyl resorcinol and/or cannabinoid compound(s) are delivered in a concentration of from about 1 to about 1000 μM/ml, and more preferably between about 10 to -100 μM/ml, such as between about 25 to about 75 μM/ml.

In performance of the inventive method, the compound(s) are applied topically to one or both individuals or to an item contaminated with HIV) proximate in time to contact between the two individuals (or contact between one of the individuals and a contaminated item). Desirably, the compounds are applied topically to one or both of the individuals prior to contact between them, or to an individual or a contaminated item prior to contact between an individual and the item. For example, the compound(s) can be applied topically to an individual a few or several seconds (e.g., about 5 seconds, or even about 10 seconds or more) prior to contact between the two individuals or about one or a few minutes (e.g., longer than about 30 seconds, such as one or two minutes, or even five minutes or more, such as at least about 15 minutes or more). Indeed, suitable protection can be achieved by topical application of the compound(s) as much as half and hour or one or several hours prior to contact between the individuals. In some applications, infection can be successfully attenuated if the resorcinol derivative compound(s) and/or cannabinoid(s) is applied topically prior to or after contact, such as within a few minutes (such as within a few hours, or possibly within about one or a few days or so) of contact with a first individual or item contaminated with HIV. Indeed, although a composition containing the compound(s) is typically applied just prior to intercourse, the compound(s) ability to remain in the vaginal vault for at least one to three days is advantageous over the long term especially since certain cannabinoids can have a high non-specific binding to the vaginal epithelium resulting in increased vaginal stratification and a mucoid cell layer overlying the stratified epithelium, leading to increased mucus production, which, in itself, can provide additional protection.

To effectively deliver the alkyl resorcinol and/or cannabinoid compounds, they can be formulated in any desirable manner for topical application to the desired tissue. For example, the compound(s) can be formulated into a solution or suspension (e.g., in water or oil) or in a gel, cream, salve, or other fluid or semi-fluid formulation suitable for topical application. Alternatively, the compounds can be formulated into a composition to be used in conjunction with other devices, preferably barrier devices (e.g., condoms, sponges, diaphragms, etc.). Methods of formulating compositions for use alone or in conjunction with such barrier devices are well-known in the art, and any of them can be employed as desired.

While, for use in the inventive method, the alkyl resorcinol and/or cannabinoid compounds can be formulated in any appropriate and desired manner, the invention also provides a composition suitable for topical application tissue that comprises at least one alkyl resorcinol and/or cannabinoid compounds and a water insoluble bioadhesive polymer as a hydrogel. Bioadhesive polymers are polymers that can adhere onto a biological substrate. Hydrogels are hydrophilic matrices capable of swelling and not dissolving in an aqueous media as water. The resorcinol and/or cannabinoid comound(s) can be loaded into these bioadhesive polymers, or hydrogels, so that as water is absorbed into the matrix, chain relaxation occurs and drug molecules are released through the spaces or channels within the hydrogel network.

Within the inventive composition, the active agent (i.e., the alkyl resorcinol, cannabinoid, or combination thereof) typically represents at least about 1%, such as at least about 2% or at least about 5% of the composition, and can represent as much as about 20% of the composition, more typically as much as about 15% or up to about 10% of the composition. Desirably, as noted above, the alkyl resorcinol and/or cannabinoid compound(s) are present in a concentration of from about 1 μM/ml to about 1000 μM/ml, and more preferably between about 10 μM/ml to about 100 μM/ml, such as between about 25 μM/ml to about 75 μM/ml. However, if desired, a much greater concentration of the resorcinol and/or cannabinoid compound(s) can be employed, such as up to about 100 mM/ml, or even up to about 1000 mM/ml or 5000 mM/ml.

Many bioadhesives are made of either synthetic or natural polymers. Most of the current synthetic bioadhesive polymers are either polyacrylic acid or cellulose derivatives. Representatives of polyacrylic acid-based polymers are carbopol, polycarbophil, polyacrylic acid (PAAc), polyacrylate, poly(methylvinylether-co-methacrylic) acid, poly(2-hydroxylethyl methacrylate), poly(methacrylate), poly(alkylcyanoacrylate), poly(isohexylcyanoacrylate), and poly(isobutylcyanoacrylate). Cellulosics include carboxymethyl cellulose, hydroxyethol cellulose, hydroxypropyl cellulose, sodium carboxymethyl cellulose, methyl cellulose, and methylhydroxyethyl cellulose. In addition, (semi) natural bioadhesive polymers include chitosan and various gums such as guar, xanthan, gellan, carrageenan, pectin, and alginate. Finally, PHPMAm, poly(vinylpyrrolidone), and poly(vinylalcohol) can be included as synthetic bioadhesive polymers. To achieve the desired bioadherence and consitency, it is desirable for the polymer to constitute between about 0.5% and about 5% of the composition, more typically between about 1% and about 3% of the composition by weight.

A preferred polymer for use in this invention is Polycarbophil, U.S.P., which commercially available from B. F. Goodrich Speciality Polymers of Cleveland, Ohio under the trade name NOVEON® AA-1 USP. This water insoluble polymer has an apparent pKa of approximately 4.5, picks up 60-100 times its weight in water. It is a synthetic, non-absorbed, non-toxic, substance, which is stable to elevated temperatures and high oxygen content. Gels containing polycarbophil have been demonstrated to remain on vaginal tissue for 3-4 days and serve as a platform for the delivery of agents such as progesterone. Since the cannabinoids have been compared to the steroids in structure the use of polycarbophil as the principle functioning polymer is desirable.

Other bioadhesive polymers that can be used in conjunction with NOVEON® AA-1 are Noveon® Carbopol® 934P NF, Carbopol 974P NF, and Carbopol 971P NF. Carbopol 934P NF polymer has been used in oral suspensions and tablets worldwide since the mid 1960s. In the past ten years, Noveon, Inc. has designed two new products polymerized in ethyl acetate, as toxicologically preferred alternatives to Carbopol 934P NF polymer. Carbopol 974P NF has similar rheological properties to Carbopol 934P NF: both are highly crosslinked polymers which produce semisolid formulations with very short flow rheology. Short flow rheology can be characterized as a gelled consistency similar to mayonnaise. Carbopol 971P NF is a lightly crosslinked polymer, which provides very low viscosities and excellent yield values at low usage levels. Semisolid dosage forms based on Carbopol 971 P NF polymer have a longer rheology, and will flow in a manner not unlike honey. Phase change polymers that undergo a change from liquid to semisolid also can be used. Examples of phase change polymers are poloxamer 407, sodium carboxymethycellulose, carbopol, hyaluronic acid, or xanthum gum.

Typically, bioadhesive polymers are fabricated by a polymeric reaction of a polymer or pre-polymer and a cross-linking agent. Suitable cross-linking agents include divinyl glycol, divinylbenzene, N,N-diallylacrylamide, 3,4dihydroxy-1,5-hexadiene, 2,5-dimethyl-1,5-hexadiene and similar agents. The cross-linking agent should be present at such an amount as to provide enough bioadhesion to allow the system to remain attached to the target epithelial surfaces for a sufficient time to allow the desired dosing to take place. Also in most preferred practice, the bioadhesive contains about 0.05% to about 2% by weight cross-linking agent, although it may contain about 0.01% to about 10% by weight cross-linking agent. The polymer formulation can be adjusted to control the release rate of the drugs (cannabinoids and alkyl resorcinols) by varying the amount of cross-linking agent in the polymer. For example, greater than two percent of the cross-linking agent can decrease the ability of the polymer to absorb water and swell, which may be desirably in some systems.

The rheologic properties of a gel will determine the residence time of a given drug in the formulation which in contact with the desired surface. Its release will be determined by a number of factors including drug interaction with the polymer and the partition of the drugs to micelles. The physical characteristics of the gels themselves are determined by the degree of cross-linking. For example, Carbomer 934P is a gel former that can be used in the inventive formulations, and which can be substituted by other gel formers, such as Carbomer 974P, Carbomer 980 and methyl cellulose or propyl cellulose. Although C981 and C940 differ in cross-linking density (C940 is more highly cross-linked), the release typically does not differ. Carbopol® 1342 which has a covalently bound, lipophilic modification, i.e., a long-chain (C10-C30) alkyl acrylate. It is belived to be the lipophilic interactions between the micelles and the polymer that results in the slower release from this gel. On the other hand, it has been shown that Carbomer 934P had a zero-order release in the small bowel of fasting rats.

For vaginal administration, preferably the formulation remains attached to the epithelial surfaces for a period of at least about twenty-four to about seventy-two hours. Such results may be measured clinically over various periods of time. This preferred level of bioadhesion is usually attained when the cross-linking agent is present at about 0.1 to 6.0 weight percent of the polymer, with about 1.0 to 2.0 weight percent being most preferred, as long as the appropriate level of bioadhesion results. A convenient dosage form for vaginal self-administration is a gel. Thus, a product is prepared containing 1-3% polycarbophil plus the usual formulation of excipients to produce a thick emulsion-gel. The pH of the product can be adjusted to between 2.5 and 7, more typically between 3.0-6, depending upon its intended target treatment; typical pH for vaginal application is between about 4.5 and 6.5.

The interaction of the various Carbopols and polycarbophil can influence which combination of active agents and exipients is chosen. The higher calcium binding affinity found for Carbomer compared to, for example, polycarbophil can be ascribed to their different ways of cross-linking. Polycarbophil is cross-linked by divinylglycol and to a lower degree than carbomer, which is cross-linked by allylsucrose. For example, a study of the combination of polycarbophil and Carbopol 934P showed that the calcium binding affinity of polycarbophil was observerd to be statistically significant (P<0.05) lower than for carbomer. It is, however, within the ordinary skill in the art to select an appropriate polymer and cross-linking agent suitable to the desired end-use.

The advantage that these materials possess is in their ability to provide retention of a drug at a mucosal surface for a period of time longer than that found for a simple liquid or powder system. Polymer blends such as polycarbophil and daichitosan can combine attributes of different polymers to give a superior bioadhesive. Since the cannabinoids are highly lipophilic and have been shown to have high non-specific binding to the vaginal epithelium and less to the lamina propria.

The use of dry dextran-starch microspheres should be avoided since it leads to a reversible shrinkage of the cells which ultimately leads to a physical separation of the intercellular junctions which would be undesirable in the setting of STDs, such as HIV-1, HSV-2 and bacterial pathogens.

Penetration enhancers (e.g., sodium glycocholate, sodium deoxycholate, and sodium lauryl sulfate), also can be incorporated into the inventive composition. Such agents can increase the permeability of the pharamaceutical agents across mucosa, and hence their bioavailability. Where such penetration enhancers are included, typically they constitute between about 0.5% to about 10% w/v of the composition, and more typically between about 1% and about 5% w/v of the composition; however, somewhat more or less penetration enhancer can be employed as desired.

To prepare the cannabinoids or alkyl resorcinols for incorporation into the inventive formulation, the compounds can be dissolved or emulsified in a suitable carrier, typically an oil. Dissolving 1 gm of the drugs in 1 ml. alcohol or 10 gm of the drugs in 25 ml of warmed sesame oil has been the traditional route of solubilization. However, hemp seed oil is used desirably instead of sesame oil in the inventive formulations. The preferred oils derived from hemp oil are polyunsaturated essential fatty acids: gamma-linolenic acid (c 18:3w6) (GLA) and its metabolite (1-6%) dihomo-gamma-linolenic acid or DGLA (C20:3w6), LA linoleic acid (C18:2w6) (50-70%), LNA Linolenic (C18:3w3) (15-25%) which can have anti-oxidant effects. Other fatty acids can be used in the emulsifying complex: lipoic acid is both fat and water-soluble and is easily absorbed and transported across cell membranes and acts as both an extracellular and intracellular antioxidant. It forms an inclusion complex with β-cyclodextrin with a 1:1 stoichiometry. Coenzyme Q10 (ubiquinone) is another fatty acid with antioxidant effects and can be complexed with the cannabinoids and alkyl resorcinol(s). Although Tween 80 (polysorbate 80) has been used for emulsification in opthalmic preparations of cannabinoids, as an anionic detergent it is not preferred to be employed in the inventive compositions. Additionally, Tween 80 exhibits effects on uterus and oestrus cycle in the rat similar to DES. Polysorbate can be replaced with 5% polyvinylpyrrolidone (PVP) which is also useful for its suspension capabilities as well as lubricating and adhesive properties. It can be complexed with povidone as a co-polymer. Those skilled in the art will know the methods by which an emulsion is prepared prior to solubilization. The hydrophile-lipophile balance or HLB will determine the specific ratios of emulsifying agents chosen for the admixture. For example, when sodium lauryl sulfate is used as an emulsifying agent it should not be used at greater than 5% w/v to prevent any potential irritation to the vaginal mucosa. Sodium lauryl sulfate is more acid-stable and will maintain the emulsion in a pH range of 4.5 to 6.5 which is the ideal pH range of the vaginal secretions. Typically, where an emulsifying agent is included in the inventive composition, it represents between about 0.5% to about 10% of the composition, more typically between about 1% and about 5% of the composition, often about 3% by weight of the composition.

In addition to the polymeric system, optional penetration agent, and optional emulsifying agent, the inventive compositions also can include a solubilizing agent. Preferred solubilizing agents are cyclodextrins (CDs), which are oligosaccharides having 6-8 glucopyranose units connected in a ring. Within the context of the present invention, the term “cyclodextrins” includes cyclodextrins and their derivatives, e.g., ether, ester and amide derivatives. Suitable cyclodextrins include alpha-cyclodextrin, beta-cyclodextrin and gamma-cyclodextrin, 2-hydroxy-propyl-b-cyclodextrin (2-HP b-CD), methyl-beta-cyclodextrin (2,6-DM14-b-CD), sulfobutylether b-cyclodextrin (SBE-b-CD), polymer-beta-cyclodextrin. Their cyclic structure gives cyclodextrins a hydrophobic cavity. Cyclodextrins typically are used to increase the water solubility of drugs by complexing them into the hydrophobic cavity of cyclodextrin. Less polar drug molecules and hydrophobic drugs can enter these cavities, forming an inclusion complex. The inclusion complexation could enhance both the solubility and the stability of the included drug molecules. The distribution of the solutes between micelles can be influenced by cyclodextrins. Controlling the degree of substitution is important in balancing water solubility and complexing capability. For example, the introduction of a methyl substituent at the 2- and 6-positions appears to improve the inclusion of a variety of drugs to the CD cavity. Binding constants are on average 5 times greater for 2,6-DM 14-b-CD than for b-CD however due to the potential renal toxicity should not be used systemically (Thompson DO). Methyl groups seem to increase the hydrophobicity of the CD cavity as well as increase the solubility of the derivative over that of the parent CD. The extent of methylation is important in optimizing complexation. Two commercial preparations of (2HP)-b-CD, Encapsin and Molecusol®, recognized the need for this compromise and have substitution levels that provide a balance between solubility and complexation. Encapsin® and Molecusol® have MDS values of approximately 4 and 8, respectively.

Sulfobutylether b-CD: An optimal anionic CD. SBE-b-CD preparation exhibit good water solubilities and effective complexation characteristics at all levels of substitution but a hepta-substituted preparation is the optimal specification for a commercial SBE b-CD derivative. This level of substitution effectively eliminates residual b-CD in the product most economically. SBE7.-b-CD (Captisol) has high intrinsic aqueous solubility (>50% wt/vol) and exhibits binding capacities comparable to unsubstituted O-CD but often better than HP-β-CD. Its inability to form 1:2 complexes may contribute to potential safety benefits. This is marketed as Captisol by Cydex. Captisol is not a penetration enhancer, which is good for a membrane active drug.

There is a good correlation between sizes of cyclodextrin cavities and the cross-sectional area of the polymers which should permit the delivery of the CD solubilized drug to reach the targeted tissue. Examples in the cannabinoid literature confirm the feasibility of solubilizing Δ⁸-THC with a cyclodextrin: HE-211 solution was prepared at a 100 uM concentration in 10% β-hydroxypropyl cyclodextrin solution. This may not translate into a molar ratio of 1:1. PCT application 99/32107 shows that β-hydroxypropyl cyclodextrin was used with THC. An inclusion complex comprising a b cyclodextrin, hydroxypropyl-b-cyclodextrin or SBE-β-CD in molar ratio of 1:1 or 1:2 with cannabinoid or alkyl resorcinol is desirable. A further embodiment comprises sufficient cyclodextrin to form an inclusion complex comprising gamma-cyclodextrin, hydroxypropyl-cyclodextrin and polymer-beta-cyclodextrin in molar ratio cannabinoid or alkyl resorcinol:cyclodextrin of 1:2 or 1:1. A still further embodiment comprises polymer-beta-cyclodextrin with molecular weight between 4000 and 4500 as the agent capable of forming an inclusion complex with the cannabinoid and alkyl resorcinol. The weight ratio of solubilizing agent to cannabinoid is typically in the range of 100:1 to 5:1, preferably 30:1 to 10:1. Thus, where cyclodextrins are employed in the composition as solubilizing agents, they can represent between about 1% and about 25% of the composition, more typically between about 3% and about 20% of the composition, such as between about 5% and about 15% of the composition.

Compositions useful in the present invention can also contain one or more pharmaceutically or cosmetically acceptable additives that are referred to herein as adjuvants that typically assist in providing extended shelf life and customer acceptance of a hygiene product. Exemplary adjuvants include preservatives, tissue toners, tissue conditioning agents, tissue feel enhancers, emollients, lubricating oils (e.g., lipids), emulsifying agents, humectants, coloring agents, and odor providing agents (odorants).

Typical preservatives known for use with feminine hygiene products include alcohol, ascorbyl palmitate, benzoic acid, butylated hydroxyanisole, butylated, hydroxytoluene, chlorobutanol, ethylenediamine, ethylparaben, ethyl vanillin, glycerin, methylparaben, monothioglycerol, phenol, phenylethyl alcohol, phenylmercuric nitrate, propylparaben, sassafras oil, sodium benzoate, sodium formaldehyde sulfoxylate, sodium metabisulfite,sorbic acid, sulfur dioxide, maleic acid, and propyl gallate. Obviously, to the extent any of the foregoing preservatives are irritating to the vagina, less irritating preservatives should be chosen.

Typical emollients known for use with feminine hygiene products, which are useful herein, are generally bland, fatty or oleaginous substances including castor oil, sulfated castor oil, cocoa butter, coconut oil, cold cream, com oil, cotton-seed ail, rosewater ointment (also known as cold cream), combinations of sodium lauryl sulfate, propylene glycol and stairwell alcohol, sesame oil, theobroma oil, myristyl alcohol and shark liver oil.

Typical lubricating agents or oils known for use with feminine hygiene products, which are useful herein, are petrolatum, white or yellow wax, coca butter, oleic acid, olive oil, jojoba oil, paraffin, starch glycerite, lanolin, hydrophilic petrolatum, mineral oil, acetyl alcohol, glyceryl monostearate, stearic acid, polyethylene glycols, polyoxyl 40 stearate, polysorbate, silicone elastomer, cbolesterol and higher molecular weight lipids. Where present, typically such lubricating agents constitute between about 0.5% and about 5% of the composition by weight, such as between about 1% and about 3% of the composition.

Emollients and lubricants provide hygiene products with the appropriate slip, tactile feel and rub-in properties to enhance the ease of usage and to encourage the consumer to use the product more liberally and more frequently. Certain quaternary compounds allow substances like petrolatum be combined with glycerine and in personal-care products without feeling greasy. The petrolatum-glycerine combinations especially effective in alleviating dry skin. Typical emulsifying agents known for use with feminine an hygiene products, which are useful herein, are sodium alginate, carbomer, sodium carboxymethylcellulose, carrageenan, gelatin, hydroxyethylcellulose, hydroxypropylcellulose, hydroxypropylmethylcellulose, octoxynol-9, oleyl alcohol, polyvinyl alcohol, povidone, sodium lauryl sulfate, sorbitan esters, stairwell alcohol, tragacanth, and xanthan gum. Emulsifying agents are used to produce oil-in-water emulsions and can be classified into three types: monomolecular, multimolecular and solid particle. Known monomolecular emulsifying agents include potassium laurate, polyoxyethylene sorbitan monooleate. Multimolecular emulsifying agents include acacia and gelatin. Solid particle emulsifying agents include bentonite, graphite and magnesium hydroxide. Emulsifying agents can also be classified chemically into anionic, cationic and nonionic.

Typical humectants known for use with feminine hygiene product agents, which are useful herein, are glycerin, propylene glycol, pyrrolidone carboxylic acid, sodium lactate, urea, and certain natural lipid mixtures. Other known humectants include certain proteins, gelatin, hyaluronic acid, vitamins and some natural ingredients. Some of the proteins used are collagen, elastin, placental proteins and proteins from epidermal tissues of mammals are also used.

The composition also can contain one or more preservatives, such as are commonly-employed in the art. A preferred preservative is methyl- and propylparabens and sorbic acid, but others can be used as desired.

One combination of the above compounds—polycarbophil, Carbopols, cyclodextrins, 5% polyvinylpyrrolidone with the combined emulsion admixture of the novel cannabinoids and/or resorcinol with lipoic acid and Coenzyme Q10 as an emulsion—can produce a suitable composition for use in the inventive method. The exact formulation can vary depending on the delivery mode, i.e. gel, suppository, gelatin capsule or slow delivery device. However, in general, the ranges of the excipients on a weight percent basis (unless as otherwise noted) can be as follows:

| | |
|---|---|
| Solvent | Purified water: 40 to 80% |
| Emulsifying agents | Polyvinylprrilone with povidone: 1 to 5% |
| | Lipoic acid or Coenzyme Q 10 or -linoleic acid or |
| | hemp oil: 1 to 3% |
| Active Agent | Cannabinoid or alkyl resorcinol: 1 to 10% (to |
| | provide from 10 to 100 μM/ml) |
| Solubilizing agents | cyclodextrins: 5 to 15% |
| Bioadhesives: | NOVEON ® AA-1 (polycarbophil) (1 to 3%) |
| | Carbopol (a gel forming polymer) (1 to 3%) |
| | Chitosan |
| Permeability enhancers | Sodium glycocholate (1 to 5% w/v) |
| | Sodium lauryl sulfate (1 to 5% w/v) |
| Preservatives | Methylparaben (0.5 to 2%) |
| Lubricating agents | Propylene glycol or Silicone elastomer, Dow |
| | Corning (1 to 3%) |

Because sexual contaxt is a primary mode of transmission of HIV, one preferred mode of use of the inventive composition is vaginally. For such application, the composition can be applied in an amount of about 0.01 to about 5 mg/cm², such as between about 0.05 and about 3 mg/cm² and of contacted vagina cells. Given that the vagina has, on average, an interior surface area of about 40 cm², a dosage of about 1.75 grams to 2.5 grams of the inventive composition can be employed vaginally. However, more or less of the composition can be used as desired. In fact, application to the vaginal epithelium can be and preferably is excess of that needed to provide microbicidal activity.

In order to deliver the product of the inventive method to the vagina and cervix, and suitable method can be employed, e.g., by a cervical cap or diaphragm containing a gel, by insertion of a suppository or gel cap administered by a plunger, as a gel or solution administered by a catheter attached to a container, vaginal sponge, disposable squeeze bottle or needleless syringe, or by a douche or other suitable instrument such as a fenestrated tampon like device or contained in a male condom. Any of the devices which are introduced into the vagina for the delivery of the compositions can be coated by a material which will promote the release of the composition from the internal storage chamber. Alternatively, the inventive product can be delivered by a soft elastic capsule, which can dissolve in the vaginal environment. The gelation can be further plasticized by the addition of glycerin, sorbitol, or a similar polyol. Of a major concern is the source of the gelatin. If it is animal source, religious concerns may prevent its use particularly in Hindu and Muslim countries, unless it were derived from fish. Kosher preparations are available and synthetic, animal free gel caps may become available.

### PREPARATORY EXAMPLES

### Preparatory Example 1

A mixture of 2,6-dimethoxyphenol (73.4 g, 0.48 mole), 2,6-dimethyl-2-heptanol (69.0 g, 0.48 mole) and methanesulfonic acid (95 mL) was stirred at 50° C. for 3 h and then at room temperature overnight. The mixture was poured over ice-water (600 mL) with stirring. The mixture was extracted with CH₂Cl₂ (2×200 mL). The extracts were washed with water, saturated aqueous NaHCO₃, saturated aqueous sodium chloride solution and dried over anhydrous Na₂SO₄. The solution was concentrated under reduced pressure to obtain the product as an oil (130 g, 96%). Analysis of this substance (MS (FAB) m/z 281 (MH)+; 1H NMR (CDCl3) δ0.80 (d, 6H), 1.0-1.1 (m, 4H), 1.27 (s, 6H), 1.40-1.60 (m, 3H), 3.89 (s, 6H), 5.36 (s, 1H), 6.54 (s, 2H)) revealed it to be 4-(1,1,5-trimethylhexyl)-2,6-dimethoxyphenol (referred to hereinafter as IMG-502):

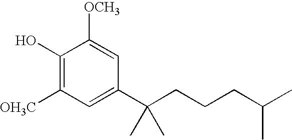

### Preparatory Example 2

A solution of crude 4-(1,1,5-trimethylhexyl)-2,6-dimethoxyphenol from Example 1 (130 g, 0.46 mole) in dry CCl₄ (100 mL) was cooled in ice-bath and diethyl phosphite (70 mL, 0.54 mole) was added. To the stirred mixture triethylamine (75 mL, 0.54 mole) was added dropwise at such a rate as to maintain the temperature of the reaction mixture below 10° C. The reaction mixture was stirred in the ice-bath for 2 h and at room temperature overnight. The mixture was then diluted with CH2Cl2 (200 mL), washed with water, 4N aqueous NaOH (100 mL), 1N aqueous HCl (125 mL), water and saturated aqueous sodium chloride solution. The extracts were dried over anhydrous Na₂SO₄ and concentrated under reduced pressure. The crude product was purified by chromatography over a column of silica using cyclohexane:EtOAc (7:1 to 3:1 gradient) as the eluent to obtain 103 g (54%) of the product as a colorless waxy oil. Analysis of this substance (MS (FAB) m/z 417 (MH)+. 1H NMR (CDCl3) δ0.81 (d, 6H), 1.0-1.1 (m, 4H), 1.26 (s, 6H), 1.35-1.6 (m, 9H), 3.86 (s, 6H), 4.25-4.38 (m, 4H), 6.53 (s, 2H)) revealed it to be 4-(1,1,5-trimethylhexyl)-2,6-dimethoxyphenyl diethyl phosphate:

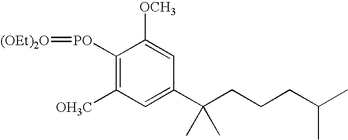

### Preparatory Example 3

A solution of 4-(1,1,5-trimethylhexyl)-2,6-dimethoxyphenyl diethyl phosphate from Example 2 (82 g, 0.197 mole) in Et₂O (175 mL) and THF (35 mL) was added slowly to liquid ammonia (450 mL) contained in a 3-neck vessel fitted with mechanical stirrer, thermometer, dry ice condenser and a pressure equalizing addition funnel while adding small freshly cut pieces of lithium wire (2.8 g, 0.40 g-atom) at such a rate as to maintain a blue color. The reaction mixture was stirred further for an hour and then quenched by the addition of saturated aqueous NH₄Cl (22 mL). Ether (220 mL) was added and the ammonia was allowed to evaporate overnight. The residue was treated with water (220 mL). The layers were separated and the ether layer was washed with 4N NaOH (200 mL), water (2×200 mL) and saturated aqueous sodium chloride solution. The organic extracts were dried (MgSO4) and concentrated under reduced pressure. The crude product was purified by chromatography over a column of silica using cyclohexane:EtOAc (95:5) as the eluent to obtain 43 g (83%) of the product as a colorless oil. Analysis of this substance (MS (FAB) m/z 265 (MH)+; 1H NMR (CDCl3) δ0.80 (d, 6H), 1.00-1.10 (m, 4H), 1.26 (s, 6H), 1.4-1.6 (m, 3H), 3.79 (s, 6H), 6.30 (m, 1H), 6.49 (m, 2H)) revealed it to be 4-(1,1,5-trimethylhexyl)-2,6-dimethoxybenzene (referred to hereinafter as IMG-503):

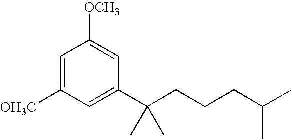

### Preparatory Example 4

A solution of 4-(1,1,5-trimethylhexyl)-2,6-dimethoxybenzene from Example 3 (10 g, 0.038 mole) in anhydrous CH₂Cl₂ (100 mL) was cooled in ice-bath and was treated dropwise with a solution of boron tribromide in CH₂Cl₂ (100 mL of 1M solution, 0.10 mole) over a period of 1 h. The mixture was stirred in the cold bath for 2 h and then at room temperature overnight. The reaction mixture was cooled in ice-bath and cautiously treated with water (100 mL). The resulting mixture was diluted with CH₂Cl₂ (100 mL) and treated with half-saturated aqueous sodium bicarbonate solution. The layers were separated, the organic layer was concentrated to half volume under reduced pressure and extracted with 2N aqueous NaOH (2×75 mL). The aqueous alkaline extract was cooled and acidified to pH 3.0 with 1N aqueous HCl. The acidified mixture was extracted with Et₂O (2×100 mL). The ether layer was washed with saturated aqueous sodium chloride solution, dried over anhydrous MgSO₄ and concentrated under reduced pressure. The crude product thus obtained was purified by chromatography over a column of silica using cyclohexane:EtOAc (8:1 to 4:1 gradient) as the eluent to obtain 8.0 g (90%) of the product as colorless crystalline solid. Analysis of this substance (Mp 95-96° C. MS (FAB) m/z 237 (MH)+; 1H NMR (CDCl3) δ0.80 (d, 6H), 1.00-1.10 (m, 4H), 1.23 (s, 6H), 1.40-1.58 (m, 3H), 4.65 (s, 2H), 6.17 (m, 1H), 6.38 (m, 2H)) revealed it to be 5-(1,1,5-trimethylhexyl) resorcinol (referred to hereinafter as IMG-501):

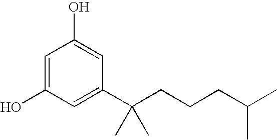

### Preparatory Example 5

A solution of 4-(1,1,5-trimethylhexyl) resorcinol from Example 4 (2 g, 0.0076 mole) in anhydrous CH₂Cl₂ (10 mL) was cooled in ice-bath and was treated dropwise with a solution of boron tribromide in CH₂Cl₂ (2.6 mL of 1M solution, 0.0026 mole). The mixture was stirred in the cold bath for 2 h and then at room temperature overnight. The mixture was cooled in ice-bath and cautiously treated with water (10 mL) followed by saturated aqueous sodium bicarbonate (5 mL). The organic layer was separated, dried over MgSO₄ and concentrated under reduced pressure. The residue was purified by chromatography over a column of silica using cyclohexane:EtOAc (8:1 to 4:1 gradient) as the eluent to obtain 0.364 g (19%) of the product as a colorless oil. Analysis of this substance (MS (FAB) m/z 251 (MH)+; 1H NMR (CDCl3) δ0.80 (d, 6H), 1.00-1.10 (m, 4H), 1.24 (s, 6H), 1.4-1.6 (m, 3H), 3.78 (s, 3H), 4.67 (s, 1H), 6.23 (m, 1H), 6.40 (m, 1H), 6.47 (m, 1H)) revealed it to be 3-methoxy-5-(1,1,5-trimethylhexyl)phenol (referred to hereinafter as IMG-504):

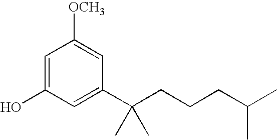

### Preparatory Example 6

To solution of crude 4-(1,1,5-trimethylhexyl)-2,6-dimethoxyphenol from Example 1 (0.19 g, 0.68 mmol) in dry THF (6 mL) was added iodomethane (0.78 g, 5.4 mmol). The mixture was treated with 60% dispersion of sodium hydride in mineral oil (0.06 g, 1.5 mmol) under nitrogen atmosphere. The mixture was stirred at room temperature for 24 h and then concentrated under reduced pressure. The residue was treated with ether (20 mL). Water (5 mL) was added cautiously. The layers were separated, the ether layer was washed with water (5 mL), dried (MgSO₄) and concentrated under reduced pressure. The crude product was purified by chromatography over a column of sillica using cyclohexane/EtOAc 6:1 as the eluent to obtain 0.17 g (85%) of the product. Analysis of this substance (MS (FAB) m/z 295 (MH)+. 1H NMR (CDCl3) δ0.81 (d, 6H), 1.0-1.2 (m, 4H), 1.28 (s, 6H), 1.40-1.60 (m, 3H), 3.84 (s, 3H), 3.87 (s, 6H), 6.53 (s, 2H)) revealed it to be 1-(1,1,5-Trimethylhexyl)-3,4,5-trimethoxybenzene (referred to hereinafter as IMG-507):

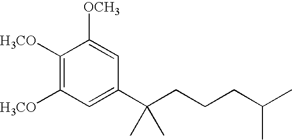

### Preparatory Example 7

A solution of 1-(1,1,5-Trimethylhexyl)-3,4,5-trimethoxybenzene from Example 6 (0.344 g, 1.5 mmol) and geraniol (0.348 g, 1.5 mmol) and p-toluenesulfonic acid (0.03 g) in dry benzene (50 mL) was heated at reflux for 2 h. The mixture was concentrated to dryness. The crude product was purified by chromatography over preparative thick layer plates (2×0.25 mm) using cyclohexane:EtOAc 5:1 as the developing solvent followed by chromatography over a column of silica using cylcohexane/EtOAc 95:5 as the eluent to obtain 0.107 g (20%) of the product. Analysis of the compound (MS (FAB) m/z 373 (MH)+. 1H NMR (CDCl3) δ0.80 (d, 6H), 1.0-1.2 (m, 4H), 1.21 (s, 6H), 1.31 (s, 3H), 1.4-1.51 (m, 2H), 1.52-1.7 (m, 9H), 1.7-1.9 (m, 2H), 2.0-2.15 (m, 2H), 2.60 (t, 2H), 4.56 (s, 1H), 5.1 (m, 1H), 6.31 (s, 1H), 6.39 (s, 1H)) revealed it to be 3,4-Dihydro-2-methyl-2-(4-methyl-3-pentenyl)-7-(1,1,5-trimethylhexyl)-2H-1-benzopyran-5-ol (referred to hereinafter as IMG-508):

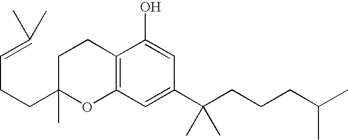

### Preparatory Example 8

A solution of 5-(1,1,5-trimethylhexyl) resorcinol (0.472 g, 2 mmol), p-menth-2-ene-1,8-diol (0.30 g, 2.1 mmol) and p-toluenesulfonic acid (0.084 g) in dry benzene (25 mL) was refluxed under a Dean-Stark trap for 4 h. The mixture was cooled to room temperature and treated with saturated aqueous sodium bicarbonate (25 mL). The layers were separated. The aqueous layer was extracted with benzene. The combined organic extracts were dried (MgSO4) and concentrated under reduced pressure. The crude product was chromatographed over a column of silica gel using cyclohexane/EtOAc 95:5 as the eluent to obtain 0.22 g (30%) of the product. Analysis of the product (MS (FAB) m/z 371 (MH)+. 1H NMR (CDCl3) δ0.80 (d, 6H), 1.00-1.10 (m, 4H), 1.11 (s, 3H), 1.21 (s, 6H), 1.39 (s, 3H), 1.4-1.52 (m, 3H), 1.71 (s, 3H), 1.75-1.95 (m, 3H), 2.1-2.2 (m, 1H), 2.62-2.73 (m, 1H), 3.12-3.25 (m, 1H), 4.61 (s, 1H), 5.4-5.5 (m, 1H), 6.23 (s, 1H), 6.39 (s, 1H)) revealed it to be 3-Norpentyl-3-(1,1,5-trimethylhexyl)-Δ8-tetrahydrocannabinol (referred to hereinafter as IMG-509):

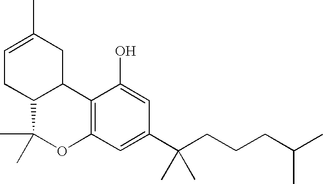

### Preparatory Example 9

A solution of 4-(1,1,5-trimethylhexyl)-2,6-dimethoxyphenol (10 g, 35.7 mmol) in dry pyridine (70 mL) was cooled to 0° C. To the stirred solution was added dropwise trifluoromethanesulfonic anhydride (11 g, 39 mmol). After the addition was complete, the reaction mixture was allowed to warm to room temperature and stir at room temperature overnight under argon. To the mixture was added an additional quantity of trifluromethanesulfonic anhydride (1.7 g, 6 mmol) and stirred for 2 h at room temperature. The mixture was concentrated under reduced pressure to remove most of the pyridine. The residue was treated with cold water (100 mL) and extracted with CH₂Cl₂ (3×50 mL). The organic extracts were washed with 1N HCl and brine, dried and concentrated under reduced pressure to obtain an orange syrup (14 g, 95%). The triflate thus obtained was used as such in the next step.

A mixture of the above triflate (10 g, 23.3 mmol), anhydrous lithium chloride (8.3 g, 196 mmol), triphenylphosphine (3.83 g, 14.6 mmol) and dichlorobis(triphenylphosphine)palladium (II) (1.8 g, 2.6 mmol) in anhydrous DMF (110 mL) was placed in a stainless steel pressure vessel under an atmosphere of nitrogen. To this mixture was added tetramethyltin (10 g, 56 mmol) and a few mg of 2,6-di-tert-butyl-4-methylphenol. The mixture was heated in an oil bath at 120° C. for 24 h. An additional quantity of tetramethyltin (5.5 g, 19 mmol) and a few crystals of 2,6-di-tert-butyl-4-methylphenol were added and the mixture was heated at 130° C. for 24 h. The mixture was cooled to room temperature and was filtered through a pad of celite to remove the palladium catalyst. The filtrate was concentrated under reduced pressure to ¼ the volume and filtered to remove yellow solid. The filtrate was further concentrated to near dryness. The residue was dissolved in CH₂Cl₂ (200 mL) and washed successively with 1.5 N HCl (5×100 mL), saturated aqueous potassium fluoride (5×50 mL), and brine. The organic layer was dried (MgSO₄) and concentrated under reduced pressure to obtain dark oil. This was purified by chromatography over a column of silica using cyclohexane/CH₂Cl₂ gradient (97:3 to 90:10) to obtain 1.82 g (27%) of the dimethoxy methyl compound. This product was utilized as such in the next step.

A solution of the above dimethoxy compound (1 g, 3.6 mmol) in CH₂Cl₂ (20 mL) was cooled to 0° C. and treated dropwise with 1M solution of BBr3 in CH₂Cl₂ (7.2 mL, 7.2 mmol). The mixture was stirred in the cold bath for 2 h and then at room temperature overnight. The reaction mixture was cooled in an ice bath and diluted with half-saturated aqueous sodium bicarbonate solution (20 mL). The mixture was diluted with CH₂Cl₂ (25 mL), and the layers were separated. The organic extracts were dried (MgSO₄) and the solvent was removed under reduced pressure to obtain a beige solid which was purified by chromatography over a column of silica using cyclohexane/EtOAc 95:5 as the eluent to obtain 0.41 g (46%) of the product. Analysis of the product (Mp 145-147° C. MS (FAB) m/z 251 (MH)+. 1H NMR (CDCl3) δ0.80 (d, 6H), 1.00-1.10 (m, 4H), 1.21 (s, 6H), 1.40-1.55 (m, 3H), 2.11 (s, 3H), 2.07 (s, 2H), 6.37 (s, 2H)) revealed it to be 2-Methyl-5-(1,1,5-trimethylhexyl)resorcinol (referred to hereinafter as IMG-510):

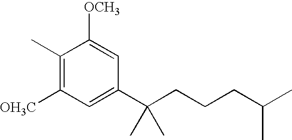

### Preparatory Example 10

A mixture of 3-norpentyl-3-(1,1,5-trimethylhexyl)-Δ8-tetrahydrocannabinol (1.4 g, 3.8 mmol) and elemental sulfur (0.3g, 0.5 mmol) was placed in a test tube and heated in a sand bath at 240-260° C. for 3 h. The crude product was purified by chromatography over a column of silica using cyclohexane/EtOAc 97:3 as the eluent to obtain 0.7 g (51%) of the product. Analysis of the product (MS (FAB) m/z 367 (MH)+. 1H NMR (CDCl3) δ0.79 (d, 6H), 1.00-1.11 (m, 4H), 1.25 (s, 6H), 1.38-1.58 (m, 3H), 1.60 (s, 6H), 2.39 (s, 3H), 5.09 (s, 1H), 6.41 (s, 1H), 6.56 (s, 1H), 7.05 (d, 1H), 7.15 (d, 1H), 8.16 (s, 1H)) revealed it to be 3-Norpentyl-3-(1,1,5-trimethylhexyl)cannabinol (referred to hereinafter as IMG-511):

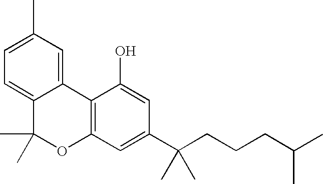

### Experimental Example 1

The studies described below encompass 5 specific IMG compounds (507 to 511) and cannabidiol and their activity in various assays. All work, except the classical attachment and fusion assays with engineered HeLa cells, was performed using peripheral blood mononuclear cells (PBMCs). All compounds were assessed in time of addition assays, for inhibition of HIV-1 attachment and fusion during standard 6 day antiviral assays in PBMCs. During the course of these studies additional experiments were performed or modified procedures devised when problems were encountered with individual efforts.

The IMG compounds compounds were prepared as solutions in 100% DMSO. Cannabidiol was purchased from Sigma Chemical (St Louis, Mo.), and solubilized in 100% DMSO. All stocks in DMSO were stored frozen at −20° C., and thawed immediately before use. Light precautions were used during stock preparation and assay set-up to minimize the exposure of the solubilized compounds to ambient light during handling.

PBMC Isolation and Blasting:

Peripheral blood monocular cells (PBMCs) were obtained from normal hepatitis and HIV-1 negative donors by ficoll hypaque gradient separation. The mononuclear cells were washed to remove residual separation media, counted, viability determined and resuspended in IPMI 1-640 medium supplemented with 15% FBS (heat inactivated), 2 mM L-glutamine, 100 U/mL penicillin, 100 μg/mL streptomycin, and 10 μg/mL gentamycin with 2 μg/mL phytohemagluttin (PHA) at 1×10⁶ cells/mL. The cells were cultured for 48 to 72 h at 37° C., 5% CO₂. Following incubation, cells were collected by centrifugation, washed and resuspended in RPMI 1-640 supplemented with 15% FBS (heat inactivated), 2 mM L-glutamine, 100 U/mL penicillin, 100 μg/mL streptomycin, and 10 μg/mL gentamycin with 20 U/mL recombinant IL-2 (R & D Systems, Minneapolis, Minn.). IL-2 was included in the culture medium to maintain the cell division initiated by the PHA mitogenic stimulation, and promote optimal growing conditions for the PBMCs. The cultures were then maintained until use by culture volume change with fresh IL-2 containing medium every 3 days.

PBMC Assay:

Human peripheral blood mononuclear cells (PBMCs) from a minimum of 2 donors, that have been blasted with PHA and IL-2, were counted, viability determined by Trypan Blue dye exclusion and mixed in equal ratios. Pooled donors were used to minimize the variability observed between individual donors which results from quantitative and qualitative differences in HIV infection and overall response to the PHA and IL-2 of primary lymphocyte populations. The cells were resuspended at 1×106 cells /mL in RPMI 1640 without phenol red supplemented with 15% Fetal Bovine Serum (heat inactivated), 2 mM L-glutamine, 100 U/mL penicillin, 100 μg/mL streptomycin, 10 μg/mL gentamycin and IL-2 (20 U/mL, R&D Systems, Minneapolis, Minn.). Fifty microliters of cells were then distributed to the inner 60 wells of a 96 well round bottom microtiter culture plate in a standard format developed by the Infectious Disease Research department of Southern Research Institute. Each plate contains cell control wells (cells only), virus control wells (cells plus virus), and experimental wells (drug plus cells plus virus). Alternatively, cells were cultured in T25 cm² or T75 cm² tissue culture flasks at a starting density of 1×10⁶ cells per ml. Serially diluted compounds were added to the microtiter plate or tissue culture flask followed by the appropriate pre-titered strain of HIV-1. For virus replication inhibition experiments the HIV-1 strain RoJo was used. This is a low passage presumable subtype B pediatric isolate with a syncytium inducing (SI) phenotype in MT-2 cells isolated by Southern Research Institute personnel. All samples were assayed in triplicate where possible with an accompanying determination of compound toxicity. The final volume per well in the microtiter plates was 200 μL. The final volume in the tissue culture flask varied depending upon experimental design and flask size. Assays were incubated for 6 days in a humidified atmosphere at 37° C., 5% CO², after which supernatants were collected, for analysis of RT activity and cell viability by MTS dye reduction. Culture were also examined microscopically, any abnormalities noted, and in the case of tissue culture flasks cell counts and viability determined by Trypan Blue Dye exclusion.

PBMC Time of Addition Assays/Attachment Assays

Time of addition assays were performed with PBMCs in two ways. For both assays, PBMCs were isolated and cultured under the conditions as described above. Time of addition assays were carried out in either a tissue culture flask format (T-75 cm²) or in microtiter wells. In both cases due to the lack of plastic adherence and heterogeneity of the PBMC population for susceptibility to infection, it was not possible to use complete removal of virus following a timed adsorption to synchronize the infection and limit the assay to a single round of infection. Therefore, serial washing by partial removal of the media was used to reduce the concentration of virus and compound (if required) in the assay. Compound addition in relationship to antiviral activity was either assessed over a short addition interval immediately before or after virus addition or for up to 72 h post infection. For all assays virus replication was assessed at 6 days of culture by determining RT expression in cell-free supernatants. In the tissue culture flask format cells were counted and Trypan Blue Dye exclusion used to monitor compound cytotoxicity. Cell viability in the 96 well microtiter plate format was determined by MTS dye reduction. AZT was used as a positive control.

MTS Staining for Cell Viability:

At assay termination the assay plates were stained with the soluble tetrazolium-based dye MTS (CellTiter96® Reagent Promega) to determine cell viability and quantify compound toxicity. MTS is metabolized by the mitochondria enzymes of metabolically active cells to a soluble formazan product, allowing the rapid quantitative analysis cell viability and compound cytotoxicity. This reagent is a single stable solution that does not require preparation before use. At termination of the assay 20 μL of MTS reagent was added per well and incubated for 4 h at 37° C. Adhesive plate sealers were used in place of the lids, the sealed plate was inverted several times to mix the soluble formazan product and the plate was read spectrophotometrically at 490 nm with a Molecular Devices Vmax plate reader.

Reverse Transcriptase Assay for Culture Supernatants:

Reverse transcriptase activity was measured in cell-free supernatants. Tritiated thymidine triphosphate (NEN) (TTP) was resuspended in distilled H₂O at 5 Ci/mL. Poly rA and oligo dT were prepared as a stock solution which was kept at −20° C. The RT reaction buffer was prepared fresh on a daily basis and consists of 125 μL 1.0 M EGTA, 125 μL dH₂O, 110 μL 10% SDS, 50 μL 1.0 M Tris (pH 7.4), 50 μL 1.0 M DTT, and 40 μL 1.0 M MgCl₂. These three solutions were mixed together in a ratio of 2 parts TTP, 1 part poly rA:oligo dT, and 1 part reaction buffer. Ten microliters of this reaction mixture was placed in a round bottom microtiter plate and 15 μL of virus containing supernatant was added and mixed. The-plate was incubated at 37° C. in a water bath with a solid support to prevent submersion of the plate and incubated for 60 minutes. Following reaction, the reaction volume was spotted onto pieces of DE81 paper, washed 5 times for 5 minutes each in a 5% sodium phosphate buffer, 2 times for 1 minute each in distilled water, 2 times for 1 minute each in 70% ethanol, and then dried. Opti-Fluor O was added to each sample and incorporated radioactivity was quantitated utilizing a Wallac 1450 Microbetaplus liquid scintillation counter.

P24 Antigen ELISA:

ELISA kits were purchased from Coulter Electronics. The assay was performed according to the manufacturer's instructions. Control curves were generated in each assay to accurately quantitate the amount of p24 antigen in each sample. For experiments using cell lysates. Fifty thousand (5×10⁴) viable cells were lysed in Coulter ELISA buffer and subjected to 1 round of freeze/thawed to liberate trapped p24 and p24 detected per manufacturers instructions. HIV capsid protein (p24) was quantitated spectrophotometrically at 450 nm using a Molecular Devices Vmax plate reader using a standard curve. Final concentrations were calculated from the optical density values using the Molecular Devices Soft Max software package.

Attachment Assay:

The attachment assay was performed with the HeLa CD4 LTR β-gal cells available from the AIDS Research and Reference Reagent Repository. HeLa cells do not express cell surface CD4, express the HIV coreceptor CXCR4 and are not infectable by HIV-1 unless CD4 is present. HeLa CD4 LTR β-gal cells express cell surface CD4 and contain an LTR β-galactosidase reporter construct. Upon infection either the Tat protein incorporated into the virion or new Tat produced following virus integration and transcription trans-activates the LTR β-gal reporter, leading to expression of the β-galactosidase enzyme. HeLa CD4 LTR β-gal cells are routinely cultured with the required selection antibiotics and screened for mycoplasma contamination. Twenty-four h prior to initiation of the assay cells were trypsinized, counted and 1×10⁴ cells placed in a 0.2 cm well in media without selection antibiotics. Media was removed at 24 h and compound in media placed on the cells and incubated for 15 to 30 min at 37° C. A known titer of virus was then added to the wells and the incubation continued for 1 h. At the end of the incubation the wells were washed 6 times with media and the culture continued for 48 h. At 48 h the wells were washed one time with PBS and β-galactosidase enzyme expression determined by chemiluminescence per manufacturers instructions (Tropix Gal-screen™, Tropix, Bedford, Mass.). Compound toxicity was monitored on sister plates using MTS dye reduction.

The attachment assay with PBMCs was performed as follows. Briefly, 1×10⁶ PHA/IL-2 PBMCs were incubated for 0, 2, 4 and 24 hours with IMG compounds and cannabidiol in polypropylene tubes. A pre-titered amount of HIV-1 (96USHIPS7 strain, obtained from the AIDS Research and Reference Reagent Program ) was added in the presence of compound and incubated for 3 h at 37° C. After incubation the cultures were washed 3 times (400×g, 10 min, 4° C.) with media (1:25,000 dilution of input p24), the cell pellets lysed with p24 lysing buffer (Coulter) and p24 content determined by ELISA.

Fusion Assay.

This assay uses the HeLa CD4 LTR β-gal and HL2-3 cells available from the AIDS Research and Reference Reagent Repository. The fusion assay utilizes HeLa CD4 LTR β-gal cells with HL2/3 cells as a fusion partner via the interaction of HIV gp 120 with CD4. HL2/3 cells express HIV-1 Env on their cell surface and contain an HIV-1 Tat expression cassette. Upon syncytium formation and plasma membrane fusion, cytoplasmic mixing of the HL2/3 and HeLa CD4 LTR b-gal cell contents results in the transactivation of the LTR and β-Galactosidase expression is then directly related to inhibition of cell membrane fusion. Thus, these two mechanistic assays can be used to confirm and identify a virus entry-based target of the compound. Additionally, because the attachment assay uses cell-free virus this assay can be used to show applicability to cell-free virus transmission.

Both cell lines were maintained as suggested by the AIDS Research and Reference Reagent Repository. Twenty-four h prior to initiation of the assay cells were trypsinized, counted and 5×10³ cells placed in a 0.2 cm well in media without selection antibiotics. To initiate the fusion assay HeLa CD4 LTR β-gal (5×10³ cells per 0.2 cm well) were incubated with compound for 1 h at 37° C. HL2/3 cells (5×10³) were added to the 0.2 cm well and the incubation continued for 48 h. At 48 h the wells were washed 1 time with PBS and β-galactosidase enzyme expression determined by chemiluminescence per manufacturers instructions (Tropix Gal-screen™, Tropix, Bedford, Mass.). Compound toxicity was monitored on sister plates using MTS dye reduction.

Data Analysis:

IC₅₀ (50%, inhibition of virus replication), I₅₀ (inhibitory concentration 50%) TC₅₀ (50% reduction in cell viability) and a therapeutic index (TI, IC₅₀/TC₅₀) were calculated for all antiviral assessments using a software package.

Results and Discussion

Inhibition of Virus Attachment and Fusion

One potential mechanism of action for the IMG compounds and cannabidiol was inhibition of virus entry via non-specific down regulation of HIV-1 coreceptors following interaction of the compounds with cannabinoid receptors, or through non-specific membrane effects preventing the co-localization of co-receptors. Therefore, the effect of these compounds was assessed using assays designed to monitor inhibition of HIV attachment and fusion.

Two experiments using HeLa CD4 LTR β-gal cells are summarized in Table 1. In these experiments standard attachment assays were performed either with virus added 15 min after compound addition or following pre-treatment of the cells for 4 or 24 h with the compounds. If inhibition of HIV replication was due to non-specific loss of HIV coreceptors following cross-talk from the cannabinoid receptors; then these two time intervals should identify this effect. The 4 h incubation would have been sufficient for removal of the coreceptors by cross-talk and before recycling had replaced them. Thus the compounds would have been identified as attachment inhibitors. The 24 h pre-incubation is sufficient time for G-coupled receptor de-sensitization to occur and re-expression of the HIV coreceptors. Thus, the compounds should have had no effect on virus attachment, if non-specific coreceptor modulation was their mechanism of action. If the compounds were active this would suggest other potential antiviral targets, but not eliminate virus entry.

The IMG compounds (507-510) and cannabidiol had a variable effect on HeLa CD4 LTR β-gal cell viability (see Table 1). In the first experiment, the compounds did not alter cell viability, while in the second cyotoxicity appeared to correlate with exposure time. Because this is a 48 h assay, variability with cytotoxic compound is often observed and is dependent upon the density and health of the cells at the initiation of the assay. Therefore the results indicate that all the compounds are potentially altering the viability of the HeLa CD4 LTR β-gal cells. In addition to the effect on cell viability several compounds suppressed β-galactosidase production below background (cells alone+compounds: IMG507, ING508, IMG511 and cannabidiol at all times and IMG509 and IMG510 following 24 h exposure). In addition, transient suppression of virus replication at lower concentrations of compounds was observed consistently with IMG507 and at specific compound addition times for IMG509 (0 h) and IMG510 (4 h). These artifacts were consistent throughout two independent experiments and were not found in the positive control assays using Chicago Sky Blue (CSB). Additionally, the assays performed with CSB met all internal assays validation criteria for an acceptable assay. Therefore, a conclusion can be drawn that the compounds interact with HeLa CD4 LTR β-gal cells altering their ability to induce β-galactosidase either through a specific (alteration in transcription or protein production) or non-specific (cytotoxic) pathway.

In addition to assessing inhibition of attachment with HeLa CD4 LTR β-gal cells a virus fusion assay was perfoed using HeLa CD4 LTR β-gal and HL2/3 cells. No timed addition of compound was employed, but rather the assay was performed to verify the effects of the compounds on the HeLa CD4 LTR β-gal cells β-galactosidase expression. Table 2 summarizes these data and shows that although the compound effects in the fusion assay on the HeLa CD4 LTR β-gal cells are not as pronounced as the attachment assay, the effects are still present.

TABLE 1
Effect of the IMG Compounds and Cannabidiol in the HeLa CD4 LTR β-gal
HIV-1 Attachment Assay.

| | | Experiment | | Experiment | | |
|---|---|---|---|---|---|---|
| | Pretreat | 1 | | 2 | | |
| Cmpd. | (h) | IC₅₀ | TC₅₀ | IC₅₀ | TC₅₀ | Comment |
| CSB (μg/ml) | 0 | 1.1 | >10 | 0.17 | >10 | Positive control |
| | 4 | 1.71 | >10 | 2.11 | >10 | |
| | 24 | 1.3 | >10 | 4.7 | >10 | |
| IMG 507 | 0 | >50 | >50 | 16 | >50 | Transient suppression lost with at lower |
| (μM) | | | | | | concentrations |
| | 4 | >50 | >50 | >50 | >50 | Transient suppression lost with at lower |
| | | | | | | concentrations |
| | 24 | >50 | >50 | >50 | >50 | Transient suppression t lost with at lower |
| | | | | | | concentrations |
| IMG508 | 0 | 16 | >50 | 0.9 | >50 | Suppression of β-galactosidase activity, |
| (μM) | | | | | | Transient suppression lost with at lower |
| | | | | | | concentrations |
| | 4 | 10.8 | >50 | 11 | 29 | Suppression of β-galactosidase activity |
| | 24 | 7.4 | >50 | 2.6 | 12.2 | Suppression of β-galactosidase activity, |
| | | | | | | Transient suppression lost with at lower |
| | | | | | | concentration |
| IMG509 | 0 | >50 | >50 | 28 | >50 | Transient suppression lost with at lower |
| (μM) | | | | | | concentrations |
| | 4 | 21 | >50 | 36 | 37 | |
| | 24 | 11.5 | >50 | 3.5 | 14.9 | Suppression of β-galactosidase activity |
| IMG510 | 0 | >50 | >50 | >50 | >50 | |
| (μM) | 4 | >50 | >50 | >50 | >50 | Transient suppression lost with at lower |
| | | | | | | concentrations |
| | 24 | 0.14 | >50 | 11.5 | 39.4 | Suppression of β-galactosidase activity |
| IMG511 | 0 | 25 | >50 | <0.16 | >50 | Suppression of β-galactosidase activity |
| (μM) | 4 | 9.5 | >50 | 8.3 | >50 | Suppression of β-galactosidase activity, |
| | | | | | | Transient suppression lost with at lower |
| | | | | | | concentrations |
| | 24 | 2.5 | >50 | 7.1 | 11.1 | Suppression of β-galactosidase activity |
| Cannabidiol | 0 | 14.1 | >50 | 1.3 | >50 | Exp. 1 suppression of β-galactosidase |
| (μM) | | | | | | activity |
| | 4 | 6.8 | >50 | 28 | 41 | Suppression of β-galactosidase activity |
| | 24 | 1.2 | >50 | 1.4 | 33 | Suppression of β-galactosidase activity |

TABLE 2
Effect of the IMG Compounds and Cannabidiol in the HeLa CD4 LTR
β-gal/HL2/3 HIV-1 Fusion Assay.

| | Antiviral | | |
|---|---|---|---|
| | Activity | | |
| Compound | IC₅₀ | TC₅₀ | Comments |
| CSB (μg/ml) | 1.75 | >10 | Positive control |
| IMG507 | >50 | >50 | |
| IMG508 | 29.9 | 36.5 | |
| IMG509 | 22 | 29.9 | Suppression of β-galactosidase |
| | | | activity |
| IMG510 | 33.6 | >50 | |
| IMG511 | 28.4 | 33.8 | Suppression of β-galactosidase |
| | | | activity |
| Cannabidiol | 19.3 | 39.8 | Suppression of β-galactosidase |
| | | | activity |

Two observations suggest antiviral activity via targets other than specific or non-specific inhibition of virus attachment/entry. The consistent inhibition of HIV attachment/entry seen in HeLa CD4 LTR β-gal cells following a 24 h pre-incubation with IMG508, IMG510, IMG511 and cannabidiol, but not IM507 and IMG509, suggests that the interaction of the compounds with the cells are different. The overall reduction in β-galactosidase activity in untreated cells also suggests either a specific anti-HIV LTR or non-specific cellular suppression of transcription/translation by the compounds.

A modified PMBC assay next was performed to address the question of whether the compounds prevented virus replication by interruption of virus entry. PBMCs pretreated with compounds for 0, 2, 4, and 24 were incubated with cell-free HIV-1 for 3 h and the excess unbound HIV removed. The assessed the amount of HIV bound to the cell was then assessed by measuring cell-associated p24 content of total cell lysates. Table 3 summarizes the results of these experiments.

Attachment assays with PBMCs often display secondary problems due to non-specific trapping of virus. As seen in Table 3 total suppression of virus attachment with 20 μg/ml dextran sulfate was 43.4%. Therefore analysis of this experiment is based upon identifying equivalent or better suppression in one of the IMG compound treated samples. All the IMG compounds and cannabidiol modulated/reduced HIV attachment to some extent (10 to 25% inhibition) when added at time 0. And, IMG508 and cannabidiol demonstrated a return to control levels of virus binding if pretreated for 24 h. These data suggest that the IMG compounds may modulate some expression of the coreceptors. However, since these reductions in binding are in the order of the magnitude seen for nevaripine (19%, RT inhibitor) these changes cannot be separated from non-specific effects within the assay. Only IMG509 and IMG510 at 15 μM pretreated for 4 h approached the suppression of virus attachment seen with the positive control dextran sulfate, 40% and 29% respectively. Since these compounds had effects equivalent to nevirapine (10 μM) when added at infection (0 h) this strongly suggests that the compounds are modulating coreceptor expression rather than blocking virus attachment directly. However the significance of these observations cannot be determined from this data alone. The potential effect of IMG509 and IMG510 on coreceptors via cannabinoid receptor cross talk could be conclusively demonstrated through loss of chemokine ligand binding following treatment of PBMCs or other cells with these compounds.

TABLE 3
HIV Attachment Assay in PBMCs

| Compounds | Time | pg p24 | % Control | Comments |
|---|---|---|---|---|
| Nevirapine | 0 | 311.7 | 81.2 | Positive control |
| (10 μM) | | | | 19% suppression |
| Dextran Sulfate | 0 | 221 | 57.6 | Positive control |
| (20 μg/ml) | | | | 43.4% suppression |
| IMG507¹ | 0 | 320 | 83.3 | No significant effect |
| | 2 | 351 | 91.4 | |
| | 4 | 293 | 76.3 | |
| | 24 | 368 | 95.8 | |
| IMG508 | 0 | 312 | 81.3 | No significant effect |
| | 2 | 328 | 85.4 | |
| | 4 | 279 | 72.7 | |
| | 24 | 428 | 111.5 | |
| IMG509 | 0 | 321 | 83.6 | 40% suppression at 4 h |
| | 2 | 314 | 81.8 | |
| | 4 | 235 | 61.2 | |
| | 24 | 297 | 77.3 | |
| IMG510 | 0 | 350 | 91.1 | 29% suppression at 4 h |
| | 2 | 329 | 85.7 | |
| | 4 | 274 | 71.4 | |
| | 24 | 339 | 88.3 | |
| IMG511 | 0 | 290 | 75.5 | No Significant effect |
| | 2 | 328 | 85.4 | |
| | 4 | 294 | 76.6 | |
| | 24 | 352 | 91.7 | |
| Cannabidiol | 0 | 317 | 82.6 | No significant effect |
| | 2 | 286 | 74.5 | |
| | 4 | 349 | 90.9 | |
| | 24 | 439 | 114.3 | |

¹All IMG compounds and cannabidiol at 15 μM

Time of Addition Studies

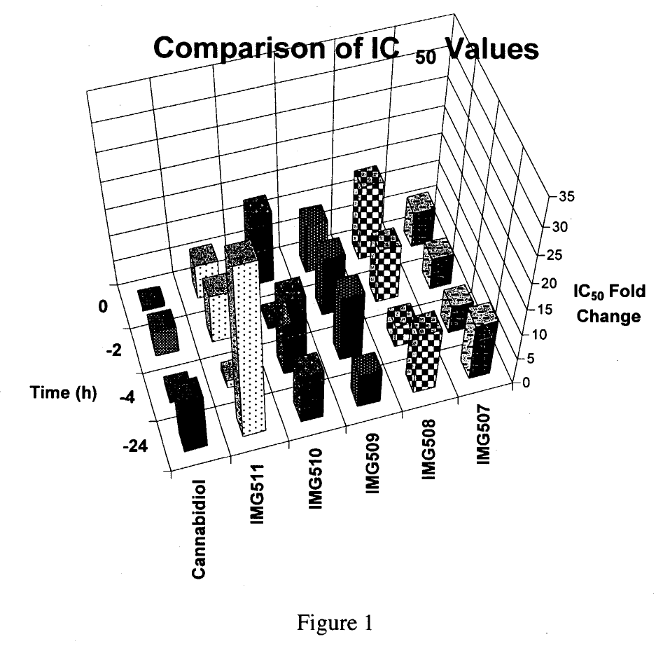

Time of addition studies with PBMCs were initially proposed based upon the hypothesis that the IMG compounds and cannabidiol were modulating antiviral activity through specific coreceptor interactions and second messenger pathways. Therefore initial studies were designed to look at the effect of pretreatment on antiviral activity. The data generated by the time of addition assay are summarized in Table 4 and FIG. 1.

TABLE 4
Time of Addition Assay with PBMC (Addition Prior to Infection)

| | Pre- | | | |
|---|---|---|---|---|
| Compounds | Treatment | | | |
| (μM) | (h) | IC₅₀ | TI | Comments |
| AZT | 0 | 0.001 | >3077 | No significant change |
| | | 3 | | in TI or IC₅₀ |
| | 2 | 0.001 | >3636 | |
| | | 1 | | |
| | 4 | 0.001 | >3636 | |
| | | 1 | | |
| | 24 | 0.003 | >1290 | |
| | | 1 | | |
| IMG507 | 0 | 6.9 | 17 | No significant change in |
| | 2 | 6.1 | 19.9 | TI or IC₅₀ |
| | 4 | 5.3 | 22.7 | |
| | 24 | 10.8 | 11.1 | |
| IMG508 | 0 | 14.2 | 3.5 | 4-fold decrease in IC₅₀ |
| | 2 | 10.4 | 4.8 | 4 h pretreatment |
| | 4 | 4.1 | 12 | |
| | 24 | 11.9 | 4.2 | |
| IMG509 | 0 | 9.3 | 5.3 | No significant change in |
| | 2 | 11 | 4.4 | TI or IC₅₀ |
| | 4 | 12.6 | 4.0 | |
| | 24 | 6.6 | 7.4 | |
| IMG510 | 0 | 13.4 | 10.4 | 9.5-fold decrease in |
| | 2 | 1.4 | 104 | IC₅₀ 2 h pretreatment |
| | 4 | 11.5 | 9.4 | |
| | 24 | 9.5 | 15 | |
| IMG511 | 0 | 6.6 | 18 | 5-fold increase in IC₅₀ |
| | 2 | 9.4 | 12.5 | with 24 h pretreatment |
| | 4 | 11.7 | 73 | |
| | 24 | 33.2 | 3.5 | |
| Cannabidiol | 0 | 0.58 | 83 | Initial 10-fold loss of |
| | 2 | 5 | 9.7 | activity at 2 , regain 3.3- |
| | 4 | 1.5 | 33 | fold at 4 h then a 17- |
| | 24 | 10.5 | 4.6 | fold increase in IC₅₀ at |
| | | | | 24 h pretreatment |
| | | | | overall |

This experiment identified some significant effects of pretreatment of PBMCs with the IMG compound and cannabidiol. The results can be divided into 4 types of responses: 1. No change, 2. Transient decrease in IC₅₀ with pretreatment, 3. Increase in IC₅₀ with pretreatment and 4. Combination of observations 2 and 3.

No Significant Change in IC₅₀

Compounds IMG507 and IMG509 showed no significant change in their IC₅₀s with pretreatment for up to 24 h. Closer examination of the IC₅₀ in FIG. 1 shows an approximately 2-fold decrease (IMG509) and increase (IMG507) at 24 h. These changes are within an expected 3-fold error for the assay for IC₅₀ as seem in the range of the IC₅₀ for AZT from 1.1 to 3.1 nM. Thus the antiviral target for these two compound is independent of early effects on the PBMCs and does not involve modulation of coreceptors. However this does not rule out a potential signaling pathway component for these compounds. It is important to note that IMG509 was able to significantly inhibit HIV cell-association with a 4 h preincubation (Table 3). This further supports the possibility that IMG509 antiviral activity is independent of cell surface and inducible or modulatable cell pathways that modify the activity of cannabinoids.

Transient Decrease in IC₅₀ with Pretreatment

IMG508 and IMG510 showed transient decreases in their IC₅₀s at 4 and 2 h, respectively. A decrease in IC₅₀ translates to an increase in antiviral potency since the compounds exerted a constant amount of cellular cyotoxicity (TC₅₀). The other pre-treatment times did not show a significant change from addition of the compounds with the virus. This pattern of activity suggests that pretreatment with IMG508 and 510 is modulating or interacting with a cellular pathway directly responsible for the potency of the antiviral response, such as a signaling pathways regulating transcriptional factor expression or compound metabolism. Optimal interaction results in a signal coding for better antiviral activity. Thus a return to initial levels indicates loss of the optimal effect by down-modulation of the receptor or signaling pathway.

The precise pathways by which IMG508 and IMG510 are producing these phenomena cannot be determined from this data. However the temporal correlation of maximal antiviral activity at 2 h and a reduction in virus attachment at 4 h for IMG410 reinforces a possible mechanism involving virus entry receptors. In contrast, since enhanced IMG508 did not correlate with attachment inhibition this suggests a mechanism of action independent of a virus entry pathway. However we cannot rule out that there are modulations of coreceptors independent of CD4, which would yield the observed attachment negative pattern.

Increase in IC₅₀ with Pretreatment

The third pattern observed was a transient increase in the IC₅₀ or a loss of antiviral potency. This was observed with IMG511 following a 24 h pretreatment. This observation suggests a loss of antiviral activity by either loss of the appropriate receptor for these compounds, receptor de-sensitization and/or induction of secondary signals working to turn off the antiviral response; thereby rendering the cells unable to respond to the compound. These results upon closer examination show IMG511 pretreatment proceeds in a simple time-dependent progression of the IC₅₀s to less activity with compound exposure, further suggesting a “shutting off” or loss of the ability to respond to the compound. This observation suggests that long term exposure to IMG511 will result in anergy to its effects.

Combination of Increased and Decreased in IC₅₀

The pattern for cannabidiol is more complex. It looses approximately 10-fold of its potency with a 2 h pre-treatment, partially regains potency (3.3 fold decrease in IC₅₀) at 4 h pretreatment and then looses any gains at 24 h (total loss in potency 17-fold). These results show that natural cannabidiol has a complex interaction with PBMCs and that the IMG analogs have through chemical means separated this complex interaction with the cell into some of its components.

In addition to displaying a number of patterns in the time of addition assays for modulation of antiviral activity two observations were made. Although it can be assumed that cells seeing addition of compound at time 0 will go through the same modulations of antiviral activity, it was observed with pre-addition the compounds retain antiviral activity in a 6 day PBMC assay with continuous exposure to the compounds. The second observation is that the IMG-derivatives have multiple antiviral mechanisms of action in PBMCs. Separation of attachment from activity modulation and multiple patterns of activity modulation suggest that these compounds are interacting with different receptors by different pathways.

The observation that the IMG compounds and cannabidiol were active through modulation of multiple antiviral targets prompted a second time of addition assay in which pre-treatment was coupled with post-treatment. Compounds added at −4, 0 and +6 h in relationship to addition of virus showed the expected modulations in IC₅₀ with pretreatment, but also showed no difference between adding the compound at infection or 6 h post infection in antiviral potency (Table 5). In these experiments AZT showed the expected enhancement of activity (4.6-fold) with a 4 h preincubation, demonstrating that it requires phosphorylation before it is active. Therefore the results at +6 h with cannabidiol and the IMG compounds suggests that although they can modulate the antiviral outcome in pre-treatment scenarios, presumably by altering receptor expression and induction of second messenger pathways, these modulations are not the primary mechanism of antiviral action. The observations following addition of compound at +6 h infection led us to assess the activity of the IMG compounds and cannabidiol in a time of addition assay which encompasses events in virus replication from virus entry to completion of reverse transcription.

TABLE 5
Time of Addition Assay (Addition at −4, 0 and +6 H Relative to Infection)

| | | Antiviral activity μM | | |
|---|---|---|---|---|
| Compound | Treatment | IC₅₀ | TC₅₀ | TI |
| AZT | −4 h pre | 0.0008 | >4 | >5000 |
| | At infection | 0.0037 | >4 | >1081 |
| | 6 h Post | 0.0046 | >4 | >869 |
| IMG507 | −4 h pre | 39.5 | >100 | >2.5 |
| | At infection | 67 | >100 | >1.5 |
| | 6 h post | 46.1 | >100 | >2.2 |
| IMG508 | −4 h pre | 6.2 | >100 | >16 |
| | At infection | 15.7 | >100 | >6.3 |
| | 6 h post | 22.5 | >100 | >4.4 |
| IMG509 | −4 h pre | 19.6 | >100 | >5.1 |
| | At infection | 17.5 | >100 | >5.7 |
| | 6 h post | 10.0 | >100 | >9.9 |
| IMG510 | −4 h pre | 17.7 | >100 | >5.66 |
| | At infection | 16.1 | >100 | >6.2 |
| | 6 h post | 14.4 | >100 | >6.9 |
| IMG511 | −4 h pre | 15.2 | >100 | >6.6 |
| | At infection | 16.9 | >100 | >5.9 |
| | 6 h post | 9.3 | >100 | >13.7 |
| Cannabidiol | −4 h pre | 12.1 | >100 | >8.3 |
| | At infection | 13.8 | >100 | >7.3 |
| | 6 h post | 12.1 | >100 | >8.3 |

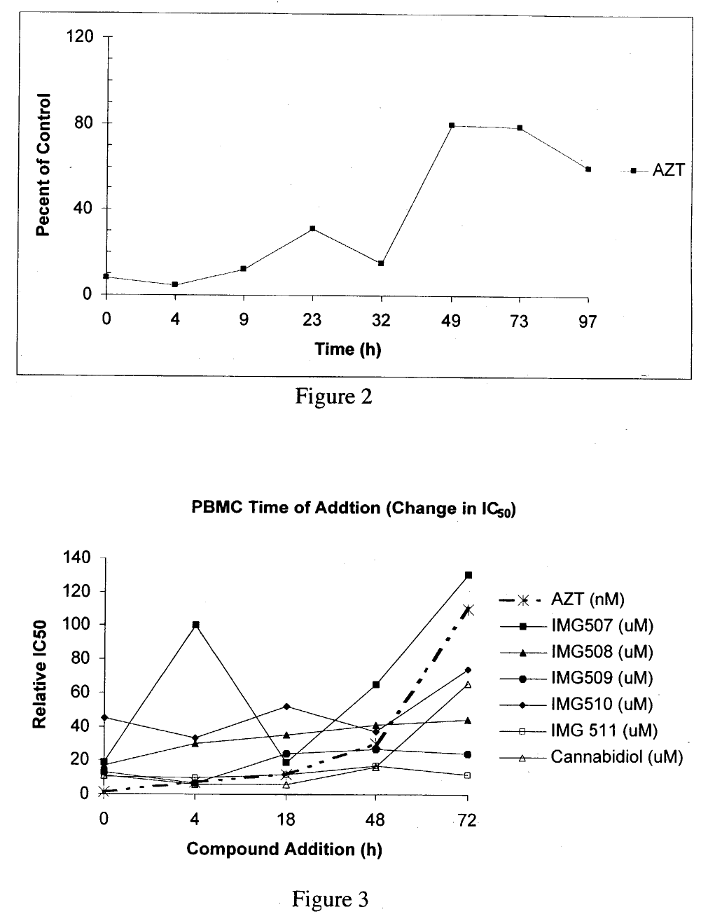

Time of addition assays using PBMC are often not as clear cut as those performed in cell line models where the initial infection can be synchronized and virus expression be assessed following a single round of infection. Typically time of addition assays to assess the role of compounds in reverse transcription or later events are performed using HeLa CD4 LTR β-gal cells. However, these compounds have secondary effects on the expression of β-galactosidase making their use problematic. Therefore we used PBMCs. The initial infection in PBMCs cannot be synchronized due to non-adherence to tissue culture substrates and the heterogeneity of the PBMC population, resulting in a frequency of initial frequency of infection of CD4+ lymphocytes of less than 10%. These characteristics prevent the simple and reproducible detection of HIV replication early in the infection. Thus PBMC-based time of addition assays must be allowed to go through multiple rounds of infection to allow sufficient virus replication to assess the effects of inhibitors. A historical example of AZT in this type of assay is shown in FIG. 2. As seen in FIG. 2 reverse transcription is complete between 32 and 48 h post infection. In this assay this means that sufficient reverse transcription has occurred that the total virus expression at 6 days (RT) allows a statistically relevant comparison with the treatment groups. Thus although additional rounds of infection are still occurring during this time the previous rounds of infection have already established sufficient virus expression that further rounds of infection do not contribute to overall virus expression.

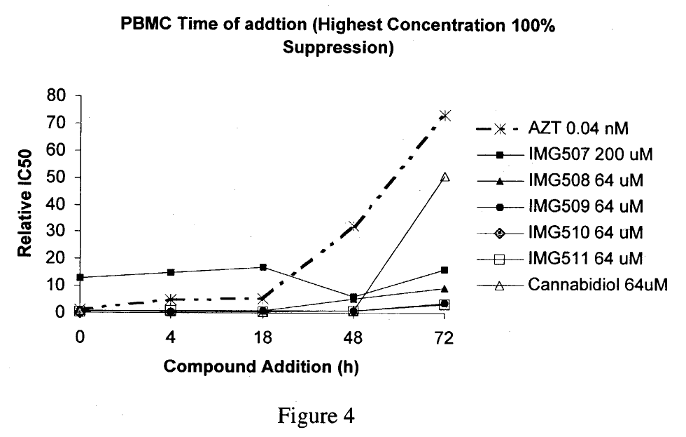

FIGS. 3 and 4 summarize the PBMC time of addition assays using addition of compounds during the interval from virus entry to completion of reverse transcription. The data is graphed in 2 formats. FIG. 3 graphs the relative IC₅₀ for each treatment at 6 days. This graph shows the relative potency of the compounds when added after virus exposure. FIG. 4 is a maximal suppression graph. This graph shows the results for each time point at the lowest concentration which results in complete suppression of virus replication when compound is added simultaneously with the virus. Comparison of the results of FIGS. 3 and 4 shows that all compounds have an antiviral target present after completion of reverse transcription. From these data IMG508, 509, 510 and 511 appear to all interact with an antiviral target other than virus entry or reverse transcription. Although the antiviral target cannot be conclusively identified, this pattern has been seen for inhibitors of HIV-1 with mechanisms of action localized to inhibition of transcription. However it also suggests there may be multiple antiviral targets or interaction with a single target with different potencies.

Additionally for IMG507 the results in FIG. 3 suggest that there are antiviral targets prior to and coinciding with reverse transcription. As identified in Table 3, IMG507 had no significant effect on virus attachment or when cells were pretreated with the compound. This suggests that the first “spike” of inhibition observed at 4 h in FIG. 3 represents an antiviral target occurring immediately after or coinciding with the virus interaction with the cell membrane/entry receptors. This is enforced by its transient nature and complete reversal at 18 h. In addition, comparison of FIGS. 3 and 4 further suggests the potential of a second or possible third antiviral target for IMG507. These experiments provide strong evidence of an antiviral target that temporally coincides with events following reverse transcription.

As suggested earlier for time of addition assays employing pretreatment of cells, cannabidiol displays a pattern of inhibition similar to the post 48 h suppression of virus replication that the IMG-congers display. However, FIG. 4 suggests that the IMG compounds are potentially more potent in their interaction with the antiviral target. Although the data suggest a possible alternative target in FIG. 4 the analysis based on IC₅₀ change in FIG. 3 would argue against an additional antiviral target.

Thus in toto the time of addition studies suggest that the IMG analogs interact with multiple antiviral targets, and are potentially more potent in their interaction with their antiviral target than cannabidiol. The pre-treatment studies go further in suggesting that the mode of interaction of the IMG congers with PBMCs may represent specific aspects of the overall interaction of cannabidiol with PBMCs. Additionally, these studies identify IMG507 as the only conger in this series with the potential to interact with virus entry targets. Finally, although IMG508-511 and cannabidiol may be able to modulate the antiviral response when pre-exposed to the cells their primary mechanism of action involves an antiviral target that is post virus entry and completion of reverse transcription.

The results discussed above reveal that the IMG compounds mediate antiviral (i.e., anti-HI) activity through an antiviral target that is independent of HIV entry and reverse transcription. Furthermore, the pretreatment studies reveal that that the compounds may interact with PBMCs via a variety of pathways inducing them with different potency and/or kinetics. Although a precise mechanism of action for the IMG compounds was not identified the studies performed here suggest that inhibition of virus replication is intimately associated with modulation of the cell cycle.

### Experimental Example 2

The studies described below encompass 5 specific IMG compounds (509, 510, and 511) and cannabidiol and their activity in virus attachment and fusion assays.

Cells and Viruses:

HIV-1IIIB, and the HeLa CD4 LTR β-gal, and HL2/3 cell lines were obtained from the NIH AIDS Research and Reference Reagent Program (Bethesda, Md.), and maintained as recommended. ME 180 cells were obtained from the American Type Culture Collection (Manassas, Va.).

Test Material Handling and Storage:

Compounds were solubilized in 100% DMSO and stored at −80° C. until tested. Frozen stocks were thawed at room temperature, pre-warmed for 15 min at 37° C. and vortexed prior to preparation of working solutions in tissue culture medium. During all stages of compound dilution and handling, compounds were protected from incidental light by opaque coverings and by storage and dilution in opaque or amber-colored tissue culture plastics. Additionally, incidental room and laminar flow tissue culture hood light exposure is controlled by reducing total fluorescent lighting in the laboratory by 50%. The final DMSO concentration is 0.25% at the highest test concentration.

Virus Attachment Assay:

This assay detects compounds that block virus attachment using HeLa CD4 LTR β-gal cells. HeLa CD4 LTR β-gal cells were cultured with selection antibiotics. Twenty-four h prior to initiation of the assay, the cells were trypsinized, counted and plated in a 0.2 cm well in media without selection antibiotics. At 24 h media is removed and compound in media placed on the cells and incubated for 15 min at 37° C. A known titer of the IIIB strain of HIV-1 was then added to the wells and the incubation continued for 1 to 2 h. At the end of the incubation the wells were washed 2 times with media and the culture continued for 40 to 48 h. At termination of the assay, media is removed and β-galactosidase enzyme expression determined by chemiluminescence per manufacturer's instructions (Tropix Gal-screen, Bedford Mass.). Compound toxicity is monitored on a sister plate by XTT or MTS dye reduction. All determinations are performed in triplicate with serial_Log10 dilution of the test materials. The virus adsorption interval of 1 to 2 h was sufficiently short that AZT, which requires phosphorylation to achieve its active tri-phosphate form (AZT-TTP), is not active in this assay.

Fusion Assay:

The fusion assay assesses the ability of compounds to block cell-to-cell fusion mediated by HIV-1 Env and CD4 expressed on separate cells. This assay is sensitive to inhibitors of both the gp 120/CD4 interaction and inhibitors of the X4 coreceptor. HeLa CD4 LTR β-gal cells were plated in microtiter wells and diluted compounds are added and allowed to incubate at 37° C. for 1 hr prior to the addition of HL2/3 cells. The incubation was then continued for 40 to 48 h, after which fusion is monitored by measurement of β-galactosidase enzyme expression, detectable by chemiluminescence (Tropix Gal-screen, Tropix, Bedford, Mass.). Compound toxicity is monitored on a sister plate using XTT or MTS dye reduction. All determinations are performed in triplicate with serial_Log10 dilution of the test materials.

Results:

The results of the virus attachment and fusion assays are presented in Table 6. The results indicate that the three IMG compounds were not inhibitory to the processes of virus attachment/entry but showed some inhibition of virus fusion.

TABLE 6

| | Attachment Assay | | | Fusion Assay | | | |
|---|---|---|---|---|---|---|---|
| Compound | IC₅₀ | TC₅₀ | TI | IC₅₀ | TC₅₀ | TI | Comments |
| Chicago Sky Blue | 0.63 | >10 | >15.8 | 0.41 | >100 | >24.39 | Active Control |
| (μg/ml) | | | 7 | | | | Compound |
| IMG 509 (μM) | 48.4 | >10 | >2.07 | 1.21 | 18.4 | 15.21 | Active Fusion, not |
| | | 0 | | | | | attachment inhibitor |
| IMG 510 (μM) | 50.8 | >10 | >1.97 | 1.36 | >100 | >73.53 | Active Fusion, not |
| | | 0 | | | | | attachment inhibitor |
| IMG 511 (μM) | 46.3 | >67 | 1.45 | 0.92 | 20.7 | 22.5 | Active Fusion, not |
| | | | | | | | attachment inhibitor |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising at least one compound selected from the group of compounds consisting of alkyl-recorcinols, cannabinoids, and combinations thereof and a bioadhesive polymer.

2. The composition of claim 1, wherein the compound is a cannabinoid selected from the group consisting of cannabinol derivatives, Δ8-THC derivatives, cannabichromene derivatives, cannabidiol derivatives, cannabigerol derivatives

3. The composition of claim 1, wherein at least one compound is IMG507, IMG508, IMG509, IMG510, or IMG511.

4. The composition of claim 1, wherein the alkyl-recorcinol, cannabinoid, or combination thereof comprises between about 1% and about 10% of the composition.

5. The composition of claim 1, wherein the alkyl-recorcinol, cannabinoid, or combination thereof is present between about 10 μM/ml to about 100 μM/ml.

6. The composition of claim 1, wherein the bioadhesive polymer comprises between about 1% and about 3% of the composition.

7. The composition of claim 1, which further comprises between about 40% and about 80% water.

8. The composition of claim 1, which further comprises between about 1% and about 5% emulsifying agent.

9. The composition of claim 8, wherein the emulsifying agent is hemp oil.

10. The composition of claim 1, which further comprises between a solubilizing agent.

11. The composition of claim 10, wherein the solubilizing agent comprises between about 5% and about 15% of the composition.

12. The composition of claim 1, which further comprises a permeability enhancer.

13. The composition of claim 12, wherein the permeability enhancer comprises between about 1% and about 5% (w/v) of the composition.

14. The composition of claim 1, which further comprises a preservative.

15. The composition of claim 1, which further comprises a lubricating agent.

16. A method of a method for preventing the transmission of HIV from a first individual harboring HIV to a second individual at risk of infection with HIV comprising topically applying at least one alkyl-recorcinol, cannabinoid, or a combination thereof to the first individual, or to the second individual, proximate in time with contact between the first individual and the second individual.

17. The method of claim 1-6, wherein the compound is a cannabinoid selected from the group consisting of cannabinol derivatives, Δ8-THC derivatives, cannabichromene derivatives, cannabidiol derivatives, cannabigerol derivatives.

18. The method of claim 1-6, wherein at least one compound is IMG507, IMG508, IMG509, IMG510, or IMG511.

19. A method of a method for preventing the transmission of HIV to an individual at risk of infection with HIV upon contact with an item contaminated with HIV comprising topically applying at least one alkyl-recorcinol, cannabinoid, or a combination thereof to the individual, proximate in time with contact between contact between the individual and the item.

20. The method of claim 19, wherein the compound is a cannabinoid selected from the group consisting of cannabinol derivatives, Δ8-THC derivatives, cannabichromene derivatives, cannabidiol derivatives, cannabigerol derivatives.

21. The method of claim 19, wherein at least one compound is IMG507, IMG508, IMG509, IMG510, or IMG511.
